(19) United States
(12) Patent Application Publication
(10) Pub. No.: US 2024/0316287 A1
(43) Pub. Date: Sep. 26, 2024

(54) DAMPED AUTOINJECTORS

(71) Applicant: FPrin, LLC, Santa Clara, CA (US)

(72) Inventors: Peter Holst, Los Altos, CA (US); Samuel Sayre, Mountain View, CA (US); Alec Lindeman, Saratoga, CA (US)

(21) Appl. No.: 18/259,999

(22) PCT Filed: Dec. 27, 2021
(86) PCT No.: PCT/US2021/065234

§ 371 Date: Jun. 29, 2023

Related U.S. Application Data
(60) Provisional application No. 63/133,405, filed on Jan. 3, 2021.

(51) Int. Cl. A61M 5/48; A61M 5/20; A61M 5/31; A61M 5/315

(52) U.S. Cl. CPC A61M 5/488; A61M 5/31511; A61M 5/3158; A61M 2005/2006; A61M 2005/3143

(57) ABSTRACT

Autoinjectors comprising an energy source mechanism, a damping mechanism and a delivery mechanism with a formulation that is to be delivered to a subject in need thereof are described. Also described are damping mechanisms that, in addition to other advantages, reduce the initial force and pressure spikes when energy is released from the energy source.

### RELATED APPLICATIONS

This application claims priority to U.S. Provisional Patent Application Ser. No. 63/133,405 filed on Jan. 3, 2021, the disclosure of which is hereby incorporated by reference to the full extent permitted by law.

### FIELD OF INVENTION

The present disclosure relates to methods and devices for reducing peak pressures in autoinjectors. Damping moderates an initial impulse when applying delivery force to an injector, for example, applying force to a syringe stopper. In one embodiment, the invention is used for injecting formulations with elevated viscosity. Methods of optimizing autoinjectors based on formulation properties and treatment requirements are also presented.

### BACKGROUND OF INVENTION

Generally, autoinjectors are injection devices that have an energy source, such as a self-contained energy source, for injecting formulations into a human or an animal, e.g., intravenous, subcutaneous, intramuscular, and intradermal. Autoinjectors can simplify the injection procedure compared to manual delivery methods by supplying the energy required for injection, as opposed to utilizing a patient or caregiver's hand strength. Many different forms of energy can be found in the prior art, e.g., electrical energy such as mains power or one or more batteries: compressed gasses including but not limited to air, nitrogen, argon; mechanical springs, including but not limited to coil springs or Belleville washers, two phase systems including but not limited to CO₂, N₂O, and fluorinated compounds such as chlorofluorocarbons, hydrofluoroalkanes, HFA134a, HF A227, difluoromethane, pentafluoromethane, R-410A, combustibles including but not limited to methanes, butanes, alcohols, and reactive chemicals including but not limited to sodium azide.

Autoinjectors can be single dose disposable devices, multi-dose disposable devices, or multi-dose refillable devices.

Preferred autoinjectors are self-contained and portable, and preferably comprise one or more self-contained power sources including, but not limited to, mechanical coil springs, batteries, compressed gasses, or two phase systems.

Injected formulations are typically liquid, but may be in any form including, but not limited to, liquids, liquid solutions, liquid suspensions, semi-solids, gels, hydrogels, solids, gasses, vapors, or powders.

It is a problem with injectors that high volume injections can cause pain at the injection site and drug leakage from the injection site. To minimize this problem, liquid injection formulations are often formulated in high concentrations. However, this often leads to elevated viscosity and thus higher forces and pressures to deliver the formulation, for example through a needle or an orifice. When these high forces are released to start an injection, there is a possibility of a very high initial force spike in the mechanism and concomitant pressure spike in the formulation. One type of autoinjector uses a plunger rod which drives a syringe stopper under a force applied by the energy source, for example a spring or compressed gas. When the plunger rod is released to start the injection, it can build up kinetic energy before contacting the syringe stopper. When the plunger rod contacts the syringe stopper, it can cause a significant pressure spike in the formulation. These spikes can cause device failures such as breakage of drug containers, high initial delivery rates, and other issues. Thus, there is a need to provide autoinjectors with a mechanism that addresses these problems.

There is also the problem that autoinjectors for use with multiple formulations will have delivery times that vary with viscosity, which can result in injections that are too fast or too slow.

Autoinjectors may be needle free. Examples of needle free autoinjectors are described in U.S. Pat. No. 8,734,384.

U.S. patent application Ser. No. 12/021,052 describes an autoinjector comprising a shock absorbing mechanism for reducing an initial transfer of force to a piston, wherein the shock absorbing mechanism is a solid cylinder into which a plunger rod is slidably received. The problem with this type of damping mechanism is that the interference fit between the ram and the cylinder must be very tightly controlled. Accordingly, this requires that the inside surface of the cylinder and the outside surface of the plunger rod must also be very tightly controlled, which makes the autoinjector difficult to manufacture. The requirement of tightly controlled interference can also result in unacceptable variation in damping with temperature.

U.S. Pat. No. 10,722,655 discloses an autoinjector with a shock absorbing element comprising a piston and a damping medium wherein the damping medium is displaced by the piston, for example through a flow channel such as an orifice. The problem with this type of damping mechanism is that it requires storage of viscous fluid, displacement of the fluid, and collection on the other side of flow channel. Another problem is that the escaped fluid must be isolated from the medication. Such flow channels are typically small and it is also a problem the damping tends to be highly dependent on the geometry of the flow channel. For example, damping through a round channel is dependent on the diameter to the fourth power so the amount of damping is sensitive to variation in feature size. It is also a problem with displacement based dampers that when used with multi-dose autoinjectors, the damping fluid must be replaced or returned to its initial configuration when the autoinjector is readied for a subsequent dose.

U.S. Patent Publication No. 20200114082 teaches a rotational damping technique. However, rotational damping mechanisms are by their nature complex. For example, U.S. Patent Publication No. 20180204082 describes torsion springs that are mounted in a perpendicular plane relative to the vertical axis of the syringe. As the torsion spring is released, torque is applied to a threaded screw or nut. A threaded screw or a threaded nut articulates with the spring and converts angular motion to linear motion and torque to force. The complexity of a rotating damper makes the autoinjector more costly and more prone to failure. Rotational schemes also introduce additional frictional loss terms that may affect delivery repeatability and require larger torque and force, and greater stored energy.

U.S. Patent Publication No. 20180207363 also discloses an autoinjector that utilizes a rotational damping technique. However, in this case, the damper is used to delay the onset of an end of dose indication, and is not used to damp a portion of the delivery mechanism.

Accordingly, there is a need in the art for an autoinjector comprising an energy source mechanism, a damping mechanism and a delivery mechanism (comprising a formulation that is to be delivered to a subject in need thereof), wherein the damping mechanism (a) reduces the initial force and pressure spikes when energy is released from the energy source, (b) reduces the variability in injection delivery times due to varying formulation properties, such as viscosity, to improve reproducibility, (c) allows the use of the autoinjector with a wider range of formulations, (d) has few or no additional components other than the damping medium, (e) has reduced dependence on the critical dimension, leading to more repeatable and easier to manufacture autoinjectors, and (f) when used with multi-dose autoinjectors, it does not result in displacement of the damping medium, and thus does not require that the damping medium be replaced or restored between doses.

### SUMMARY OF INVENTION

The present disclosure meets the above challenges, among others, and is drawn to autoinjectors for injecting formulations into a human or an animal. More specifically, the present disclosure is drawn to methods and devices to improve the reliability, size, cost, and complexity of injection systems, preferably autoinjectors, most preferably autoinjectors with self-contained energy stores. The autoinjectors can be single dose disposable devices, multi-dose disposable devices, or multi-dose refillable devices.

Preferred autoinjectors are self-contained and portable, and preferably comprise one or more self-contained power sources including, but not limited to, mechanical springs, batteries, compressed gasses, reactive chemicals, or two-phase systems.

Injected formulations are preferably liquid, but may be in any form including, but not limited to, liquids, liquid solutions, liquid suspensions, semi-solids, gels, hydrogels, solids, gasses, vapors, or powders.

In one embodiment, the autoinjector is comprised of a mechanism for reducing an initial spike, for example a pressure, force, or torque spike. Preferably, the mechanism is a damper. The damper is preferably comprised of a viscous damping medium wherein the damper creates a force that is increased with increasing relative velocity of two or more components that are in contact with the damping medium. Damping media can be Newtonian, meaning the viscosity is independent of the relative velocity of the components, or it can be non-Newtonian, meaning the viscosity increases or decreases with the relative velocity. Preferred damping media are Newtonian, or have viscosity which increases with relative velocity.

Preferably, the damper is configured such that the relative motion of the two or more components results in Couette flow. Couette flow is the flow of a viscous fluid in the space between two surfaces, one of which is moving relative to the other in a direction such that the spacing between the surfaces is essentially unchanged. The relative motion of the surfaces imposes a shear stress on the fluid, resulting in viscous losses. Couette flow dampers have significant advantages over prior art autoinjector dampers, in that they are simpler to fabricate and are more reproducible and reliable than other configurations.

Preferably, the damper comprises a damping medium between two relatively moving surfaces (i.e., one surface is moving relative to the other surface), wherein the distance between the surfaces over the areas in contact with the damping medium is relatively constant. Preferably, the relative motion is a linear motion in a direction perpendicular to the separation between the surfaces. The separated surfaces can be of any shape. Preferably, they are planar. More preferably they are cylindrical or portions of cylinders. Preferred cylinders are right circular cylinders. In a particularly preferred embodiment, the surfaces comprise sections of co-axial right circular cylinders and the relative motion is in the direction of the axes of the cylinders.

The surfaces of the damper may be dedicated components of the autoinjector, which are connected to the moving parts (one part can be moving relative to the other part(s)) that are intended to be damped through linkages, gears, springs, and the like. Preferably, at least one of the surfaces of the damper is the outer or inner surface of a moving part that is intended to be damped. In a particularly preferred embodiment, one surface of the damper is a rod, such as a plunger rod, which is used to transmit force from the energy source to a stopper in a syringe body, to move the stopper toward an outlet of the syringe body and thereby deliver the medication to the patient. The other surface of the damper can be a dedicated damper body. Preferably, the other surface is a component of the autoinjector which performs another function, including but not limited to a guide for the plunger rod, and a housing for the autoinjector.

The disadvantages of known autoinjectors include, but are not limited to:
a. possible requirement of a very high delivery forces, especially when using elevated viscosity formulations, which can lead to pressure spikes and concomitant device failures, such as breakage of drug capsules;
b. different formulation viscosities can lead to different delivery times from autoinjectors;
c. rotary dampers for autoinjectors are complex and have many moving parts and linkages;
d. for displacement-type damping media, the dimensions of the damping medium flow channel must be very small and very tightly controlled in order to have adequate and controlled damping and repeatable injection performance; and
e. for displacement-type dampers when used with multi-dose autoinjectors, the damping medium must be replaced or restored to its original configuration between doses.

The advantages of the current disclosure include, but are not limited to:
a. providing a damping mechanism which damps out force and pressure spikes during delivery from an autoinjector, resulting in lower failure rates;
b. providing a damping mechanism which reduces variability in delivery time and performance due to varying formulation properties, such as viscosity, improve reproducibility and allows the use of the autoinjector with a wider range of formulations;
c. providing a damping mechanism with few or no additional components required other than the damping medium;
d. using a damper which has reduced (for example to the first power) dependence on the critical dimension, leading to more repeatable and easier to manufacture autoinjectors; and
e. when used with multi-dose autoinjectors, it does not result in displacement of the damping medium, and thus does not require that the damping medium be replaced or restored between doses.

In one embodiment, an autoinjector comprises typical elements such as a metal coil spring to store energy for the extrusion of the contents of the syringe, a shear damper, a syringe, and elements that move along the axis of the syringe with the stopper that extrudes the contents of the syringe. In another embodiment, the shear damper could be configured to dissipate some energy, said dissipation being proportional to the velocity of the piston that is pushing the syringe stopper. Many high-viscosity compounds require high force to extrude through the preferred small needles. Because compression springs all have a characteristic spring rate and because it is preferred to complete extrusions in about 3 to 10 seconds, and because the force must be maintained at a reasonable level throughout the extrusion, it can occur that the force at the beginning of the extrusion can be so high that the syringe is damaged or broken when the piston strikes the stopper. Dampers provide a resisting force proportional to relative velocity of two elements. Certain dampers rely on Couette flow and are not manifested by displacement of the damping medium; rather they manifest as the shear of the damping medium between two elements. For example, the damping medium can be between two concentric elements where one moves with the elements that serve to deliver the formulation, and one is fixed in the reference frame of the non-moving device. The force resisting the motion of the element driving the extrusion is proportional to the dynamic viscosity of the medium, the relative velocity, the area in shear (in the case of two concentric right circular cylinder surfaces where the gap between the cylinders is small compared to the mean diameter, pi*mean diameter*length) and inversely proportional to the separation between the two elements. The shear damper could be placed in any of several locations that may offer different, more favorable, combinations of length and diameter for better usability. For example, the shear damper could be around a piston or other moving element which is aligned with a syringe rather than axially displaced. Also, the energy source could be an extension spring with tapered ends to help mitigate against challenges with buckling of compression springs. In certain embodiments, the preferred injection time (to complete extrusion) is about 2 to 15 seconds, or about 3 to 13 seconds, or about 4 to 12 seconds, or about 5 to 10 seconds, or about 8 to 10 seconds, or about 3 seconds, or about 4 seconds, or about 5 seconds, or about 6 seconds, or about 7 seconds, or about 8 seconds, or about 9 seconds, or about 10 seconds, or about 11 seconds, or about 12 seconds, or about 13 seconds, or about 14 seconds, or about 15 seconds.

Embodiment 1: According to an aspect of the disclosure, there is provided an autoinjector for injecting a formulation into a subject in need thereof, wherein the autoinjector comprises:
(a) a plunger rod comprising a first surface of the plunger rod,
(b) an energy source mechanism comprising an energy source and a trigger button,
(c) a damping mechanism comprising a second surface and a damping medium that is located between the first surface and the second surface, and
(d) a delivery section comprising a drug cartridge,
wherein upon applying a force on the trigger button, the trigger button triggers a release of energy from the energy source to a plunger rod which is configured to transmit more than about 10% of released energy to the drug cartridge and causes at least a portion of the formulation to be delivered from the drug cartridge to the subject.

Embodiment 2: The autoinjector of embodiment 1, wherein the first surface and the second surface are in contact with the damping medium and wherein after applying the force on the trigger button, a relative motion between the first surface and the second surface results in Couette flow that damps out force and pressure spikes during delivery of the formulation.

Embodiment 3: The autoinjector of embodiment 1, wherein the energy source comprises electrical energy, mechanical springs, or compressed gasses.

Embodiment 4: The autoinjector of embodiment 1, wherein the damping medium is Newtonian or non-Newtonian.

Embodiment 5: The autoinjector of embodiment 4, wherein the damping medium has a viscosity of less than about 100,000,000 cP.

Embodiment 6: The autoinjector of embodiment 1, wherein the delivery section further comprises a distal end of a plunger rod, a stopper, a needle, an orifice, and a housing with a distal end.

Embodiment 7: The autoinjector of embodiment 1, wherein the formulation is administered intravenously, subcutaneously, intramuscularly, or intradermally.

Embodiment 8: The autoinjector of embodiment 1, wherein a force is exerted by the energy source in the range of about 10 N to about 200 N.

Embodiment 9: The autoinjector of embodiment 1, wherein more than about 20% of the released energy is absorbed by the damping medium.

Embodiment 10: The autoinjector of embodiment 1, wherein between about 10% and about 90% of the released energy is absorbed by the damping medium.

Embodiment 11: The autoinjector of embodiment 1, wherein the autoinjector is a single-dose disposable device, a multi-dose disposable device, or a multi-dose refillable devices.

Embodiment 12: The autoinjector of embodiment 11, wherein the autoinjector is self-contained and portable.

Embodiment 13: The autoinjector of embodiment 12, wherein the autoinjector comprises a self-contained power source.

Embodiment 14: The autoinjector of embodiment 1, wherein the formulation is provided in liquid, liquid solution, liquid suspension, semi-solid, gel, hydrogel, solid, gas, vapor, or powder form.

Embodiment 15: The autoinjector of embodiment 14, wherein the formulation is a liquid formulation.

Embodiment 16: The autoinjector of embodiment 15, wherein the liquid formulation comprises a drug and a carrier and wherein the formulation has a viscosity of at least about 1 cP.

Embodiment 17: The autoinjector of embodiment 1, wherein the first surface and the second surface are each comprised of a section of a cylinder.

Embodiment 18: The autoinjector of embodiment 1, wherein the first surface and the second surface are each comprised of a section of a right circular cylinder, and wherein the right circular cylinders have axes which are substantially collinear and overlap.

Embodiment 19: An autoinjector for injecting a formulation into a subject in need thereof, wherein the autoinjector comprises:
(a) a plunger rod comprising a first surface of the plunger rod,
(b) an energy source mechanism comprising an energy source and a trigger button;
(c) a damping mechanism comprising a second surface and a damping medium that is located between the first surface and the second surface; and
(d) a delivery section comprising a drug cartridge; and wherein the autoinjector comprises at least one of the following characteristics:
   (i) the damping mechanism damps out force and pressure spikes during delivery from the autoinjector,
   (ii) using the autoinjector results in lower device failures such as breakage of the drug cartridges and high initial delivery rates of the formulation,
   (iii) the damping mechanism reduces variability in delivery time and performance due to varying formulation properties, such as viscosity,
   (iv) the damping mechanism improves reproducibility,
   (v) the autoinjector is used with a wider range of formulations,
   (vi) the damping mechanism has few or no additional components required other than the damping medium,
   (vii) the damping is dependent at most linearly on a critical dimension of the damping mechanism,
   (viii) the autoinjector is used as a multi-dose autoinjector and the autoinjector does not result in displacement of the damping medium,
   (ix) the damping mechanism damps out force and pressure spikes during delivery of the formulation from the autoinjector, and
   (x) the autoinjector is used as a multi-dose autoinjector, and the autoinjector does not require that the damping medium be replaced or restored between doses.

Embodiment 20: The autoinjector of embodiment 19, wherein the damping medium has a viscosity greater than or about 1000 cP, and wherein between about 10% and about 90% of the released energy is absorbed by the damping medium.

Embodiment 21: The autoinjector of embodiment 19, wherein the damping medium has a viscosity greater than or about 10,000 cP.

Embodiment 22: The autoinjector of embodiment 19, wherein the damping medium has a viscosity greater than or about 50,000 cP.

Embodiment 23: The autoinjector of embodiment 19, wherein the damping medium has a viscosity greater than or about 100,000 cP.

Embodiment 24: The autoinjector of embodiment 19, wherein the damping medium has a viscosity greater than or about 250,000 cP.

Embodiment 25: The autoinjector of embodiment 19, wherein the damping medium has a viscosity greater than or about 500,000 cP.

Embodiment 26: The autoinjector of embodiment 19, wherein more than about 20% of the released energy is absorbed by the damping medium.

Embodiment 27: The autoinjector of embodiment 20, wherein more than about 40% of the released energy is absorbed by the damping medium.

Embodiment 28: The autoinjector of embodiment 19, wherein the first surface and the second surface are each comprised of a section of a cylinder.

Embodiment 29: The autoinjector of embodiment 28, wherein the first surface and the second surface are each comprised of section of a right circular cylinder, and wherein the right circular cylinders have axes which are substantially collinear and overlap.

Embodiment 30: The autoinjector of embodiment 1, wherein the first surface and the second surface are in contact with the damping medium and wherein after applying the force on the trigger button, a relative motion between the first surface and the second surface results in Couette flow that damps out force and pressure spikes during delivery from the autoinjector.

Embodiment 31: The autoinjector of embodiment 19, wherein the energy source is comprised of electrical energy, a mechanical spring, or compressed gas.

Embodiment 32: The autoinjector of embodiment 19, wherein the damping medium is selected from Newtonian or non-Newtonian fluids.

Embodiment 33: The autoinjector of embodiment 32, wherein the damping medium has a viscosity of less than about 100,000,000 cP.

Embodiment 34: The autoinjector of embodiment 19, wherein the delivery section further comprises a distal end of plunger rod, a stopper, a needle, an orifice, and a housing with a distal end.

Embodiment 35: The autoinjector of embodiment 19, wherein the formulation is administered intravenously, subcutaneously, intramuscularly, or intradermally.

Embodiment 36: The autoinjector of embodiment 19, wherein a force is exerted by the energy source in the range of about 10 N to about 200 N.

Embodiment 37: The autoinjector of embodiment 19, wherein more than about 20% of the released energy is absorbed by the damping medium.

Embodiment 38: The autoinjector of embodiment 19, wherein between about 10% and about 90% of the released energy is absorbed by the damping medium.

Embodiment 39: The autoinjector of embodiment 19, wherein the autoinjector is a single-dose disposable device, a multi-dose disposable device, or a multi-dose refillable devices.

Embodiment 40: The autoinjector of embodiment 39, wherein the autoinjector is self-contained and portable.

Embodiment 41: The autoinjector of embodiment 40, wherein the autoinjector comprises a self-contained power source.

Embodiment 42: The autoinjector of embodiment 19, wherein the formulation is provided in liquid, liquid solution, liquid suspension, semi-solid, gel, hydrogel, solid, gas, vapor, or powder form.

Embodiment 43: The autoinjector of embodiment 42, wherein the formulation is a liquid formulation.

Embodiment 44: The autoinjector of embodiment 15, wherein the liquid formulation comprises a drug and a carrier and wherein the formulation has a viscosity of at least about 1 cP.

Embodiment 45: The autoinjector of embodiment 19, wherein the first surface and the second surface are each comprised of a section of a cylinder.

Embodiment 46: The autoinjector of embodiment 19, wherein the first surface and the second surface are each comprised of section of a right circular cylinder, and wherein the right circular cylinders have axes which are substantially collinear and overlap.

Embodiment 47: The autoinjector of embodiment 44, wherein the liquid formulation has a viscosity of greater than about 5 cP.

Embodiment 48: The autoinjector of embodiment 44, wherein the liquid formulation has a viscosity of greater than about 10 cP.

Embodiment 49: The autoinjector of embodiment 43, wherein the liquid formulation has a viscosity of greater than about 20 cP.

Embodiment 50: The autoinjector of embodiment 43, wherein the liquid formulation has a viscosity of greater than about 50 cP.

Embodiment 51: The autoinjector of embodiment 43, wherein the liquid formulation has a viscosity of greater than or about 100 cP.

Embodiment 52: A method of optimizing the autoinjector of any of the preceding claims for use with a specific formulation, the method comprises:
(a) supplying a multiplicity of damping mechanism components each of which comprises a second surface, wherein each of the damping mechanism components can be installed in the autoinjector without making any other changes to the mechanical design of the autoinjector, and wherein the damping mechanism components in the multiplicity differ from each other in that when installed in the autoinjector they define a range of average first gaps between the first surface and the second surface;
(b) selecting one of the damping mechanisms;
(c) installing the damping mechanism selected in step (b) in the autoinjector; and
(d) applying a force on the trigger button which triggers a release of energy from the energy source to the plunger rod, wherein the plunger rod is configured to transmit between about 10% and about 90% of released energy to the drug cartridge, and wherein the transmission causes at least a portion of the formulation to be delivered from the drug cartridge to the subject.

Embodiment 53: The method of embodiment 52, wherein the optimization is based on at least one component of the list comprising: formulation viscosity, formulation volume, needle gauge, needle length, and delivery time.

Embodiment 54: The method of embodiment 52, wherein optimization further comprises changing one or more of the parameters chosen from a list comprising: energy contained in the energy source, the power provided by the energy source, the viscosity of the damping medium, a second gap between two components of the autoinjector, volume of the drug cartridge, needle gauge, and needle length.

Embodiment 55: A method for operating the autoinjector of embodiment 6 or 34 wherein the method comprises the following steps:
(a) a patient or a caregiver presses the distal end of the housing with the orifice against a target injection site and presses the trigger button releasing a spring which drives the plunger rod forward,
(b) the plunger rod moves forward relative to the housing,
(c) Couette shear flow sets up in the damping medium,
(d) the plunger rod contacts the stopper and drives the stopper and the drug cartridge forward urging the needle through the orifice and into the target injection site,
(e) the drug cartridge bottoms out against the housing and stops, and
(f) the plunger rod advances the stopper through the drug cartridge under continued urging of the spring, delivering at least some of the formulation in the drug cartridge through the needle.

Embodiment 56: The autoinjector of embodiment 1, 19, 52 or 53, wherein the damping medium has a viscosity between about 100 and about 100,000,000 cP, between about 1000 and about 10,000,000 cP, between about 10,000 and about 1,000,000 cP, between about 25,000 and about 750,000 cP, or between about 50,000 and about 550,000 cP.

Embodiment 57: The autoinjector of embodiment 1, 19, 52 or 53, wherein the damping medium has a viscosity of less than about 100,000,000 cP, less than about 10,000,000 cP, less than about 1,000,000 cP, less than about 100,000 cP, less than about 10,000 cP, or less than about 1,000 cP.

Embodiment 58: The autoinjector of embodiment 11 or 39 wherein:
(a) the autoinjector is a multi-dose disposable autoinjector or a multi-dose refillable autoinjector,
(b) the plunger rod is a dual plunger rod, and
(c) the damping mechanism has a first end with a hole through which the plunger rod is fed and a second closed end.

Embodiment 59: The autoinjector of embodiment 58, wherein the closed end comprises an end closure that is fabricated from a rigid material or a compliant material.

Embodiment 60: The autoinjector of embodiment 58, wherein the closed end comprises an end closure that is fabricated of a rigid material and is over-molded with a compliant material.

Embodiment 61: The autoinjector of embodiment 6, 34, or 55 wherein the length of the needle varies depending on route of formulation injection.

Embodiment 62: The autoinjector of any of the preceding embodiments wherein the plunger rod achieves a maximum speed of about 0.1 m/s, about 0.5 m/s, about 1 m/s, about 1.5 m/s, about 2 m/s, about 2.5 m/s, about 3 m/s, about 3.5 m/s, about 4 m/s, about 4.5 m/s, about 5 m/s, about 5.5 m/s, about 6 m/s, about 6.5 m/s, about 7 m/s, about 7.5 m/s, about 8 m/s, about 8.5 m/s, about 9 m/s, about 9.5 m/s, about 10 m/s, about 11 m/s, about 12 m/s, about 13 m/s, about 14 m/s, about 15 m/s, about 16 m/s, about 17 m/s, about 18 m/s, about 19 m/s, about 20 m/s, about 25 m/s, about 30 m/s, about 35 m/s, about 40 m/s, about 45 m/s, about 50 m/s, about 55 m/s, about 60 m/s, about 65 m/s, about 70 m/s, about 75 m/s, about 80 m/s, about 85 m/s, about 90 m/s, about 95 m/s, or about 100 m/s prior to formulation being delivered from the drug cartridge.

Embodiment 63: The autoinjector of any of the preceding embodiments wherein the formulation has a dose of less than about 200 mg, less than about 150 mg, less than about 100 mg, less than about 50, less than about 10 mg, less than about 5 mg, or less than about 1 mg.

Embodiment 64: The autoinjector of any of the preceding embodiments wherein the formulation has an active ingredient with a concentration of less than 500 mg/ml, less than 100 mg/ml, less than 50 mg/ml, or less than 20 mg/ml.

Embodiment 65: The autoinjector of any of the preceding embodiments wherein the formulation has a dose volume from about 0.05 ml to about 100 ml, from about 0.05 ml to about 10 ml, from about 0.25 ml to about 5 ml, from about 0.5 to about 2 ml, from about 1 ml to about 3 ml, from about 1 ml to about 5 ml, from about 1 ml to about 8 ml, from about 3 ml to about 5 ml, from about 3 ml to about 7 ml, from about 3 ml to about 9 ml, from about 5 ml to about 7 ml, from about 5 ml to about 9 ml, or from about 7 ml to about 9 ml.

Embodiment 66: The autoinjector of any of embodiment 1, 19, 52 or 53 wherein the formulation has a viscosity between about 1 cP and about 200 cP, between about 10 cP and about 100 cP, or between about 20 cP and about 50 cP.

Embodiment 67: The autoinjector of any of the preceding embodiments wherein the drug cartridge is filled with colony stimulating factors or a pharmaceutical product.

Embodiment 68: The autoinjector of any of the preceding embodiments wherein the drug cartridge is filled with an antibody, a polypeptide, a protein, a chemical compound or a chemical element.

These and other objects, advantages, and features of the disclosure will become apparent to those persons skilled in the art upon reading the details of the formulations and methodology as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 represents a cut-away view for an embodiment of the autoinjector of the current invention.

FIG. 2 represents an embodiment of a damping system.

FIG. 3 represents an alternate embodiment of the autoinjector of the current invention.

FIG. 4 is a graph that shows the effect of damping on flange force vs. time using the current invention with damping media of 64,000, 200,000, and 554,000 cP compared to no damping medium.

FIG. 5 is a graph of peak flange force for damped vs. undamped systems at spring preloads of 40-60 N.

FIG. 6 is a graph of the damped peak flange forces from FIG. 5.

FIG. 7 shows the impact of spring preload, formulation viscosity, and damping medium viscosity on injection time.

### DETAILED DESCRIPTION OF THE INVENTION

### A. Introduction

Before the present devices and methods are described, it is to be understood that this invention is not limited to particular devices, formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### B. Definitions

In this disclosure, the terms “pressure”, “force”, “acceleration”, “torque”, “energy”, “power” and “impulse” are defined according to classical physics. Force is equal to mass times acceleration. Pressure is equal to force per unit area. Energy is equal to force times distance. Power is energy per unit time. Acceleration is the change of velocity per unit time. Torque is a twisting force about an axis of rotation. Impulse is an amount of force required to cause a change in momentum within a certain time.

The term “2 Phase System” means a compressed gas energy source for an autoinjector which is comprised of a gas in equilibrium with its liquid state. Preferred 2 phase system are comprised of CO₂, N₂O, hydrofluoroalkanes (HFAs), chlorofluorocarbons (CFCs), and the like.

The term “autoinjector” means a mechanism for injecting a formulation into a human or animal, wherein the energy for the injection is not supplied by the hand strength of a patient or care provider. An autoinjector may have energy supplied by an external energy source, such as mains power or a compressed gas. Preferred autoinjectors are self-contained with internal energy sources such as mechanical springs, compressed gasses, and the like. Preferably, autoinjectors are small, preferably pocket size, relatively light, preferably less than 1 kg mass, and easy to operate. Autoinjectors may be single dose disposable systems which are only used once, multi-dose disposable systems which are supplied with a drug reservoir containing multiple doses and when the reservoir is exhausted the autoinjector is disposed of. Autoinjectors also may be durable devices with reservoirs which are refillable or replaceable.

The term “Belleville washer” means a component of a mechanical spring power source for an autoinjector that is comprised of a washer that is not flat but rather is cupped, with the edge of the hole at the center displaced from the perimeter. Belleville washer springs may be comprised essentially only of a single Belleville washer, but are preferably comprised of 2 or more, 3 or more, 4 or more, 5 or more or 6 or more Belleville washers arranged in an alternating up/down configuration where the centers, then the perimeters, are touching. Preferably, the Belleville washer spring is comprised of a multiplicity of Belleville washers with a guiding mechanism such as but not limited to a central rod and/or a housing.

The term “coefficient of dynamic friction” means the coefficient of friction between two bodies that are in contact but moving (i.e., sliding) relative to each other.

The term “coefficient of static friction” means the coefficient of friction between two bodies that are not moving relative to each other. The coefficient of static of friction is generally larger than the coefficient of dynamic friction, and thus it generally takes a higher force to start a body sliding in contact with another body than it takes to maintain the sliding velocity.

The term “collinear” means that a set of points are on the same line. Two line segments are collinear when the line segments are extended infinitely in both directions and they become the same line.

The term “combustion system” means a compressed gas energy source for an autoinjector that is comprised of a solid, liquid, gas, or fluid that generates the pressurized gas, or compresses the gas, via chemically reacting with oxygen from the air.

The term “compressed gas” means an energy source for an autoinjector which contains a compressed gas. Compressed gas energy sources may be supplied containing essentially only a compressed gas, such as nitrogen, helium, or argon. Alternatively, the compressed gas energy source may be a 2 phase system, or be comprised of a combustible or a reactive chemical wherein the compressed gas is generated at the time of injection.

The term “Couette flow” means the flow of a viscous fluid in the space between two surfaces, one of which is moving tangentially relative to the other. The relative motion of the surfaces imposes a shear stress on the fluid.

The term “cylinder” means a rod or tube of constant cross-sectional shape and area. See also Right Circular Cylinder.

The term “damping” means a resistive force that depends on relative velocity. The velocity can be angular velocity but is preferably linear velocity.

The terms “damping medium” and “damping fluid” can be used interchangeably and mean a viscous fluid in contact with moving components or surfaces (one component or surface moving relative to the other component(s) or surface(s)) to achieve damping.

The terms “damping mechanism” and “damper” can be used interchangeably and mean the mechanism by which damping is achieved using a viscous fluid that is in contact with moving components or surfaces (one component or surface moving relative to the other component(s) or surface(s)).

The term “drug cartridge” means a component of an autoinjector that contains a formulation for injection. The drug cartridge may, in the case of a liquid, suspension, gel, hydrogel, semi-solid, gas, or powdered drug, be a container, e.g., a syringe body, preferably with a stopper that closes the container wherein the stopper may be used to force the contents out of the container, and with an exit orifice through which the formulation is delivered. In the case of a solid formulation, the drug cartridge may consist essentially only of the drug formulation.

The term “Newtonian Viscosity” means viscosity which is independent of shear.

The term “Non-Newtonian Viscosity” means viscosity which changes with shear. Non-Newtonian viscosity can be shear thinning, as in the example of tomato ketchup. It can also be shear thickening, as in the example of cornstarch and water.

The term “platform system” means an autoinjector that is designed to be used with many different formulations, for example many different formulation viscosities. Preferred platform systems are also preferably designed to be used with different needle gauges and lengths. Preferably a platform system is designed to be optimized for a given formulation and needle by changing a minimum of other parameters, preferably 3 or fewer parameter, more preferably 2 or fewer parameters. For example, a platform system may be optimized by changing a force from an energy source and a damper viscosity, a damper viscosity and a damper gap, or just one of a damper viscosity or a damper gap.

The term “plunger rod” means a mechanical component of an autoinjector that transmits force and energy from an energy source to a drug cartridge.

The term “reactive chemical” means a chemical or mixture of chemicals that can form a pressurized gas to be used as an energy source for an autoinjector, without the requirement of oxygen from the air for combustion.

The term “regular polygon” means a multisided closed shape (i.e., a polygon) that has all sides of equal length. Regular polygons may be either convex or star shape. Preferred regular polygons include but are not limited to triangles, squares, pentagons, hexagons and octagons.

The term “right circular cylinder” means a cylinder with the cross-sectional shape (when the cross section is taken at a right angle to the axis of the cylinder) which is a circle.

The term “shear” refers to a strain in the structure of a substance, wherein its layers are laterally shifted in relation to each other.

The term “shear thickening” means a property of a damping medium wherein the viscosity of the damping medium is increased when the damping medium is subjected to shear.

The term “shear thinning” means a property of a damping medium wherein the viscosity of the damping medium is reduced when the damping medium is subjected to shear.

The term “stopper” means a component of a drug cartridge used to seal a drug cartridge and which may be used to force the contents of a drug cartridge out of an orifice.

The term “syringe body” means a type of drug cartridge commonly used for the injection of liquid formulations. Commercial syringe bodies are usually fabricated from glass, polypropylene, or stainless steel, and may be comprised of a syringe flange which is used to hold the syringe in place against a force applied to a stopper. A syringe body may be comprised of a fitting, such as a luer fitting, for the attachment of a hypodermic needle.

The term “syringe flange” means an extension of a syringe body normal to the axis of the syringe body—the place where the extrusion force is reacted. If using by hand, it is where the two fingers would rest. Syringe flanges preferably are made from the same material as the rest of the syringe body, more preferably the syringe flange and body are molded, machined, or otherwise fabricated as a single, continuous part.

The term “tangential motion” means motion in the direction of a tangent line to a surface. Preferred tangential motion is motion of a cylinder or portion thereof, preferably a right circular cylinder, relative to a co-axial cylinder, along a tangent line that is parallel to the axes of the cylinders.

### C. General Device Description And Operation

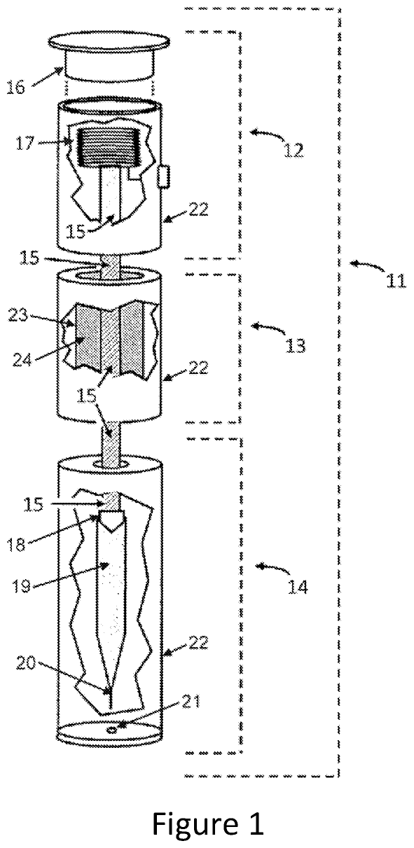

FIG. 1 shows a cut-away view of one embodiment of the invention comprising autoinjector 11 with Couette flow damping mechanism 13. The components of autoinjector 11 are separated in FIG. 1 for discussion. Energy source mechanism 12 is comprised of energy source 17 and trigger button 16. In the embodiment of FIG. 1, energy source 17 is shown as a compressed coil spring. Energy source 17 can be in general any source of energy to drive the injection, including but not limited to electrical energy such as mains power or one or more batteries; mechanical springs, including but not limited to coil springs, leaf springs or Belleville washers; or compressed gasses. Compressed gasses including but not limited to air, nitrogen, argon; can be contained in energy source 17. Energy source 17 may also contain a system for generating compressed gas. Systems for generating compressed gas include but are not limited to two phase systems including but not limited to CO₂, N₂O, and fluorinated compounds such as chlorofluorocarbons, hydrofluoroalkanes, HFA134a, HFA227, difluoromethane, pentafluoromethane, R-410A; combustibles including but not limited to methanes, butanes, and alcohols; and reactive chemicals including but not limited to sodium azide. Preferred energy sources include compressed nitrogen, compressed argon, coil springs, and CO₂. Trigger button 16 can release the energy from energy source 17 in many different ways, including but not limited to mechanically releasing a spring; releasing a compressed gas by a method including but not limited to mechanically releasing a slidable stopper, piercing a seal, breaking a seal, or actuating a valve: closing an electrical switch to power motor, solenoid, or the like, sending a signal to an electronic control system, or applying current from a second energy system such as a battery or an impactor on a piezo to an ignitor of a combustible or reactive chemical system, including but not limited to a spark gap.

Upon the triggering of autoinjector 11, energy from energy source 17 is applied to energy transmission component 15. In the embodiment of FIG. 1, the energy transmission component 15 is a plunger rod. The force can be applied to energy transmission component 15 directly, for example in the case of a mechanical spring or compressed gas, or indirectly, for example in the case of a motor or solenoid. As shown in FIG. 1, plunger rod 15 is of relatively constant cross-sectional area and shape. It may be advantageous in the case of a pressurized gas to have a section in contact with the released pressurized gas that is a different cross section, for example larger than the rest of the plunger rod, in order to increase the force exerted on and by the plunger rod.

In a preferred embodiment, a force is exerted by the energy source in the range of about 10 N to about 200 N, preferably in the range of about 15 N to about 150 N, about 20 N to about 100 N, about 30 N to about 75 N. In another preferred embodiment, the force exerted by the energy source is in the range of about 40 N to about 60 N. In yet another preferred embodiment, the force exerted by the energy source is about 10 N, about 15 N, about 20 N, about 25 N, about 30 N, about 35 N, about 40 N, about 45 N, about 50 N, about 55 N, about 60 N, about 65 N, about 70 N, about 75 N, about 80 N, about 85 N, about 90 N, about 95 N, about 100 N, about 105 N, about 110 N, about 115 N, about 120 N, about 125 N, about 130 N, about 135 N, about 140 N, about 145 N, about 150 N, about 155 N, about 160 N, about 165 N, about 170 N, about 175 N, about 180 N, about 185 N, about 190 N, about 195 N, or about 200 N.

In the embodiment where the energy source is a spring, or equivalent to a spring, for example a compressed gas, the spring constant is in the range of about 200 N/m to about 2500 N/m, preferably in the range of about 500 N/m to about 1500 N/m, more preferably in the range of about 700 N/m to about 1200 N/m. In yet another embodiment, the spring constant is about 200 N/m, about 300 N/m, about 400 N/m, about 500 N/m, about 600 N/m, about 700 N/m, about 800 N/m, about 900 N/m, about 1000 N/m, about 1100 N/m, about 1200 N/m, about 1300 N/m, about 1400 N/m, about 1500 N/m, about 1600 N/m, about 1700 N/m, about 1800 N/m, about 1900 N/m, about 2000 N/m, about 2100 N/m, about 2200 N/m, about 2300 N/m, about 2400 N/m, or about 2500 N/m.

Autoinjector 11 also includes delivery section 14. In the embodiment shown in FIG. 1, delivery section 14 is comprised of the distal end of plunger rod 15, stopper 18, drug cartridge 19, needle 20, orifice 21, and housing 22. Drug cartridge 19 can be of any material with suitable mechanical and drug contact properties. Preferred materials include but is not limited to glass, preferably borosilicate glass, or a polymer, preferably polypropylene, or a metal, preferably a stainless steel.

The operation of autoinjector 11 is as follows. The patient or caregiver presses the distal end of housing 22 with orifice 21 against the desired injection site and presses trigger button 16. This releases spring 17 and plunger rod 15, driving plunger rod 15 forward. Plunger rod 15 contacts stopper 18, and drives stopper 18 and drug cartridge 19 forward, urging needle 20 through orifice 21 and into the target injection site. Drug cartridge 19 then bottoms out against housing 22 and stops, and plunger rod 15, under the continued urging of spring 17, advances stopper 18 through drug cartridge 19, delivering the formulation in drug cartridge 19 though needle 20.

When autoinjector 11 is triggered and releases spring 17 and plunger rod 15, in the absence of any frictional losses, plunger rod 15 will accelerate to very high velocities, both before it contacts stopper 18, and as plunger rod 15 and drug cartridge 19 move forward toward orifice 21. By way of example, if spring 17 exerts a force of 100 N on a plunger of mass 1 gram over a distance of 1 cm, the mass, in the absence of frictional losses, will be accelerated to a velocity of over 14000 cm/s, approaching half the speed of sound. This can cause many issues, including breakage of components, including but not limited to drug cartridge 19, for example due to a pressure spike in formulation container 19 when plunger rod 15 strikes stopper 18, or when drug cartridge bottoms out on housing 22. Additional issues include but are not limited to too rapid delivery of the formulation causing pain and drug leakage at the injection site, recoil of the injector, and/or loud noise or vibration that may startle the subject or caregiver and cause them to withdraw autoinjector 11 before the injection is complete.

To eliminate the issues discussed above, in the embodiment of FIG. 1 autoinjector 11 comprises damping mechanism 13. Damping mechanism 13 is comprised of damping medium 24 and surface 23. Damping medium 24 is in contact with plunger rod 15 and surface 23. Surface 23 is preferably part of housing 22. Surface 23 may also be part of a mechanism to guide plunger rod 15.

Although in FIG. 1 and in the discussion that follows the damping medium is in contact with plunger rod 15, it will be understood that the damping medium can be in contact with any component of autoinjector 11 that necessarily moves during the delivery of the formulation. This moving component may be a component that is necessary for the delivery of the formulation, as in the example of plunger rod 15, or it may be connected to such a necessary component. The connection may be a rigid connection, or it may be a connection selected from but not limited to a linkage, flexure, gear, rack and pinion, or the like. In the embodiment of a single dose disposable autoinjector, the connection can also be made by the necessary component or an outcropping thereof simply pressing against the moving component. For a multi-dose autoinjector, it is preferred that the connection allows for the retraction of the moving component when the autoinjector is reset for the next dose.

Preferably there is a gap between surface 23 and plunger rod 15. Preferably the gap, as measured in a direction locally perpendicular to surface 23, is between about 0.01 mm to about 5 mm, preferably between about 0.05 mm and about 2 mm, more preferably between about 0.1 mm and about 1 mm. The gap may be selected from the following ranges: between about 0.1 mm and about 0.25 mm, between about 0.25 mm and about 0.5 mm, between about 0.5 and about 0.75 mm, and between about 0.75 mm and about 1 mm. In one embodiment, the gap is about 0.01 mm, about 0.02 mm, about 0.03 mm, about 0.04 mm, about 0.05 mm, about 0.06 mm, about 0.07 mm, about 0.08 mm, about 0.09 mm, about 0.1 mm, about 0.11 mm, about 0.12 mm, about 0.13 mm, about 0.14 mm, about 0.15 mm, about 0.16 mm, about 0.17 mm, about 0.18 mm, about 0.19 mm, about 0.2 mm, about 0.21 mm, about 0.22 mm, about 0.23 mm, about 0.24 mm, about 0.25 mm, about 0.26 mm, about 0.27 mm, about 0.28 mm, about 0.29 mm, about 0.3 mm, about 0.31 mm, about 0.32 mm, about 0.33 mm, about 0.34 mm, about 0.35 mm, about 0.36 mm, about 0.37 mm, about 0.38 mm, about 0.39 mm, about 0.4 mm, about 0.41 mm, about 0.42 mm, about 0.43 mm, about 0.44 mm, about 0.45 mm, about 0.46 mm, about 0.47 mm, about 0.48 mm, about 0.49 mm, or about 0.5 mm.

In one embodiment the area over which there is contact of damping medium 24 to one or both of plunger rod 15 and/or surface 23 is in the range of about 50 mm² to 5000 mm², preferably in the range of about 100 mm² to about 3000 mm², preferably in the range of about 250 mm² to about 2500 mm², preferably in the range of about 500 mm² to about 1500 mm². Preferred ranges for the area of contact include about 500 mm² to about 750 mm², about 750 mm² to about 1000 mm², about 1000 mm² to about 1250 mm², about 1250 mm² to about 1500 mm².

In yet another embodiment, the area over which there is contact of damping medium 24 to one or both of plunger rod 15 and/or surface 23 is about 50 mm², about 100 mm², about 150 mm², about 200 mm², about 250 mm², about 300 mm², about 350 mm², about 400 mm², about 450 mm², about 500 mm², about 550 mm², about 600 mm², about 650 mm², about 700 mm², about 750 mm², about 800 mm², about 850 mm², about 900 mm², about 950 mm², about 1000 mm², about 1050 mm², about 1100 mm², about 1150 mm², about 1200 mm², about 1250 mm², about 1300 mm², about 1350 mm², about 1400 mm², about 1450 mm², about 1500 mm², about 1550 mm², about 1600 mm², about 1650 mm², about 1700 mm², about 1750 mm², about 1800 mm², about 1850 mm², about 1900 mm², about 1950 mm², about 2000 mm², about 2050 mm², about 2100 mm², about 2150 mm², about 2200 mm², about 2250 mm², about 2300 mm², about 2350 mm², about 2400 mm², about 2450 mm², about 2500 mm², about 2700 mm², about 2900 mm², about 3000 mm², about 3200 mm², about 3400 mm², about 3600 mm², about 3800 mm², about 4000 mm², about 4200 mm², about 4400 mm², about 4600 mm², about 4800 mm², or about 5000 mm².

The functioning of damping mechanism 13 is as follows. When trigger button 16 is pressed, releasing plunger rod 15, plunger rod 15 begins to move relative to housing 22. Because plunger rod 15 is in contact with damping medium 24, in the immediate vicinity of plunger rod 15, damping medium 24 moves with plunger rod 15 relative to housing 22. However, because damping medium 24 is also in contact with surface 23, which in the embodiment of FIG. 1 comprises housing 22, in the immediate vicinity of surface 23 damping medium 24 does not move. Because of the relative movement of the opposite extremes of damping medium 24, Couette shear flow is set up in damping medium 24, resulting in viscous dissipation of the kinetic energy of plunger rod 15. In one embodiment, damping medium 24 is Newtonian. In this embodiment, the rate of energy dissipation in damping medium 24 increases linearly with the velocity of plunger rod 15. In this case, assuming a sufficiently long travel of plunger rod 15 before plunger rod 15 impacts stopper 18, or drug cartridge 19 impacts housing 22, the velocity of plunger rod 15 increases until the power transferred from energy source 17 (defined as the instantaneous force exerted on plunger rod 15 in the direction of its travel multiplied by the velocity of plunger rod 15) becomes equal to the rate of energy dissipation in the damping medium plus any other frictional dissipation. The above analysis also holds true for non-Newtonian damping media, as long as the rate of dissipation in the damping medium increases with increasing velocity, or at least decreases more slowly with velocity than the power required to achieve the increased velocity.

In a conceptually simple embodiment, the section of plunger rod 15 that is in contact with damping medium 24 is a right circular cylinder, and surface 23 is a right circular cylinder, and both cylinders have a common axis. In this case, the distance between the surfaces of the two cylinders, along a line that perpendicularly intersects the shared axis, is constant.

In the limit where this gap is small compared to the radii of the two cylinders, the force exerted on plunger rod 15 by damping medium 24 is given by:

$${F}_{damper} = \frac{μ A v}{{l}_{gap}}$$

Where A is the area of contact of damping medium 24 with plunger rod 15, v is the velocity of plunger rod 15 relative to surface 23, l_{gap} is the spacing between surface 23 and plunger rod 15, and μ is the dynamic viscosity of damping medium. One can see from the above that for a given drive force and desired rate of delivery, a damping medium may be selected based on its viscosity. The viscosity may be between about 100 and about 100,000,000 cP, between about 1000 and about 10,000,000 cP, between about 10,000 and about 1,000,000 cP, preferably between about 25,000 and about 750,000 cP, more preferably between about 50,000 and about 550,000 cP.

In one embodiment, the viscosity of the damping medium is less than about 100,000,000 cP, less than about 10,000,000 cP, less than about 1,000,000 cP, less than about 100,000 cP, less than about 10,000 cP, or less than about 1,000 cP.

In yet another embodiment, the viscosity of the damping medium is about 100 cP, about 500 cP, about 1,000 cP, about 1,500 cP, about 2,000 cP, about 2,500 cP, about 3,000 cP, about 3,500 cP, about 4,000 cP, about 4,500 cP, about 5,000 cP, about 5,500 cP, about 6,000 cP, about 6,500 cP, about 7,000 cP, about 7,500 cP, about 8,000 cP, about 8,500 cP, about 9,000 cP, about 9,500 cP, about 10,000 cP, about 15,000 cP, about 20,000 cP, about 25,000 cP, about 30,000 cP, about 35,000 cP, about 40,000 cP, about 45,000 cP, about 50,000 cP, about 64,000 cP, about 100,000 cP, about 150,000 cP, about 164,000 cP, about 200,000 cP, about 250,000 cP, about 300,000 cP, about 350,000 cP, about 400,000 cP, about 450,000 cP, about 500,000 cP, about 550,000 cP, about 554,000 cP, about 750,000 cP, about 1,000,000 cP, about 1,500,000 cP, about 2,000,000 cP, about 2,500,000 cP, about 3,000,000 cP, about 3,500,000 cP, about 4,000,000 cP, about 4,500,000 cP, about 5,000,000 cP, about 5,500,000 cP, about 6,000,000 cP, about 6,500,000 cP, about 7,000,000 cP, about 7,500,000 cP, about 8,000,000 cP, about 8,500,000 cP, about 9,000,000 cP, about 10,000,000 cP, about 20,000,000 cP, about 30,000,000 cP, about 40,000,000 cP, about 50,000,000 cP, about 60,000,000 cP, about 70,000,000 cP, about 80,000,000 cP, about 90,000,000 cP, or about 100,000,000 cP.

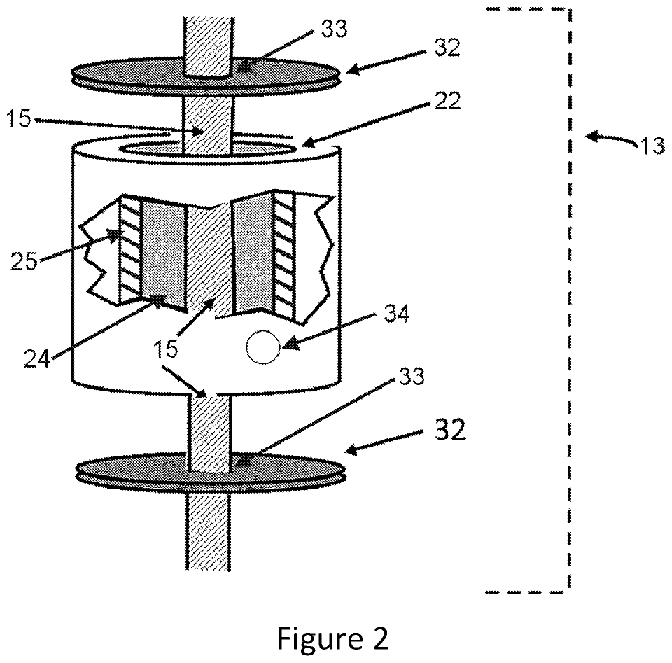

FIG. 2 presents some optional features of damper 13. In the case that the spacing between surface 23 and plunger rod 15 is larger than desired, insert 25 can be placed in the gap to make it smaller. This insert can be used to adjust the amount of damping to optimize autoinjector 11 for parameters selected from the list including but not limited to formulation viscosities, needle gauges, needle lengths, forces, desired delivery time, damping medium viscosity, etc. In a preferred embodiment, autoinjector 11 is a platform injector designed to be used for a multiplicity of drug formulations, and insert 25 is adjusted in thickness to optimize autoinjector 11 for a selected formulation, preferably a formulation viscosity, a desired needle gauge, a desired needle length, and/or a desired delivery time.

In another embodiment (not show), the cross section of plunger rod 15 is not strictly cylindrical, but instead changes as it travels through damper 13. This allows for a tailoring of the damping for different phases of the injection. In one embodiment, plunger rod 15 has a cross section which starts out relatively large during the initial run in of plunger rod 15 until plunger rod 15 contacts stopper 18, after which the cross-section of the section of plunger rod 15 which is in damper 13 is reduced, resulting in reduced or zero damping during the delivery of formulation through needle 20. In another embodiment the cross-section of the section of plunger 15 that is in damper 13 gradually is reduced during the delivery of formulation from drug cartridge 19, in this way causing a reduction in damping force to compensate for a reduction in force generated from energy store 17, as would be expected in cases where energy store 17 is, for example, a mechanical spring or a compressed gas.

In the embodiment where autoinjector 11 is a single dose disposable autoinjector, then it may be preferable to keep damper 13 open on one or both ends, for example to facilitate insertion of damping medium 24 into damper 13. However, for the embodiments where autoinjector 11 is selected from a multi-dose disposable autoinjector and a multi-dose refillable autoinjector, it is preferred that the ends of damper 13 be closed to ensure that the motion of plunger rod 15 does not cause damping medium 24 to migrate out of housing 22. This can be done utilizing end closures 32. End closures 32 are shown separated from housing 22 for clarity, but of course will be in contact with or integral with housing 22 to achieve closing. End closures 32 incorporate holes 33 through which plunger rod 15 is fed. Preferably the sections of plunger rod 15 that move through each end closure 32 are in the form of a right circular cylinder, and hole 33 are circular.

In one embodiment, end closures 32 are fabricated from a rigid material, including but not limited to a metal or a rigid plastic, and holes 33 are larger than the outside diameter of plunger rod 15, creating a gap or a tight fit that allows plunger rod 15 to pass through holes 33 over the range of manufacturing tolerances of plunger rod 15 and hole 33, but is small enough that little or no damping medium 24 exits damper 13 over the expected lifetime of autoinjector 11.

In another embodiment, end closures 32 are made of a compliant material, preferably a polymer, and hole 33 is slightly smaller than the outside diameter of plunger rod 15 prior to the insertion of plunger rod 15 and expand to essentially exactly the diameter of plunger rod 15 upon insertion of plunger rod 15, minimizing or eliminating the loss of damping medium 24 over the expected lifetime of autoinjector 11.

In yet another embodiment, end closures 32 are fabricated of a rigid material and are over-molded with a compliant material, forming holes 33.

In any embodiment where hole 33 is formed from a compliant material, hole 33 may be tapered, with one end of hole 33 slightly larger than the outside diameter of plunger rod 15, and the other end of hole 33 slightly smaller than plunger rod 15, to facilitate insertion of rod 15.

In one embodiment, one end of housing 22 is integrated with one of end closures 32. In one embodiment, plunger rod 15 is inserted through hole 33 in the end of housing 22, then damping medium 24 is injected into damper 13, and then the second end closure 32 is slid over plunger rod 15 to close damper 13 and sealed to housing 22.

In another embodiment both of end closures 32 are integral to or integrated with housing 22. Preferably, in this embodiment plunger rod 15 is partially inserted into housing 22, damping medium 24 is injected into the remaining open hole 33, and then plunger rod 15 is pushed through hole 33. In yet another embodiment, both of end closures 32 are integral to or integrated with housing 22, plunger rod 15 is inserted through both of holes 33, and damping medium 24 is injected through fill hole 34. Fill hole 34 may be subsequently capped or otherwise closed, or fill hole 34 may incorporate a check valve that stops damper medium 24 from leaking out of fill hole 34.

In some embodiments, the driving force exerted on plunger rod 15 by energy source 17, hereafter referred to as F source is not constant during the delivery. This is true of, for example, mechanical springs and single phase compressed gasses, wherein F_{drive} is reduced as plunger rod 15 moves forward. However, as long as the rate of change of F_{drive} is small, the F_{damper} will continually be equal to the instantaneous value of F_{drive}. Small rate of change of F_{drive} can be achieved with a mechanical spring if the total distance of travel of plunger rod 15 over the course of delivery is small compared to the amount that spring 17 is compressed from its equilibrium, zero force length. Similarly, small rate of change of F_{drive} can be achieved with a single phase compressed gas if the final volume containing the gas at the end of delivery is not significantly greater than the initial volume at the start of delivery.

The length of needle 20 is generally determined by the target of the injection, which in order of shortest to longest needle length is generally intradermal, subcutaneous or IV, and intramuscular. Sometimes the injection target is an internal organ or cavity and an appropriate needle length for that target is selected. The time for delivery must also be selected. Preferably, the injection time is less than about 20 seconds, more preferably less than about 10 seconds, still more preferably less than about 5 seconds, most preferably less than or about equal to 1 second. In a preferred embodiment, the injection time is between about 8 seconds and about 10 seconds, and preferably for this embodiment the injection is subcutaneous.

Once these parameters have been selected, it is possible to calculate the pressure required to deliver the selected formulation and volume through the selected needle in the selected time. In general, the hypodermic needle will be long compared to its inside diameter, for example approximately 10 to 50 times as long, and the flow is laminar, due to the small inside diameter and the often-elevated viscosity. In this case the pressure required can be predicted using the Hagen-Poiseuille equation:

$$P = \frac{μ L \dot{Q}}{2 π {ID}^{4}}$$

Where P is the pressure, u is the dynamic viscosity, L is the length of the needle, ID is the inside diameter of the needle, and Q is the volumetric flow rate. Also important is V_{plunger}, the delivery velocity of plunger rod 15, which is equal to the volume of drug cartridge 19 divided by the cross-sectional area of stopper 18 and the desired delivery time. A related quantity is v₀, the velocity of plunger rod 15 prior to the start delivery of the formulation from drug cartridge 19. Once the target pressure is determined, Fₚᵣₑₛₛᵤᵣₑ, the force that must be applied to stopper 18 to generate the required pressure, is determined by the pressure times the cross-sectional area of stopper 18 in a plane perpendicular to the direction of motion (or equivalently the cross-sectional internal area of drug cartridge 19). To this force should be added Fₛₜₒₚₚₑᵣ, the force required to overcome force due to the friction between stopper 18 and drug cartridge 19, and F_{friction}, any frictional force on plunger rod 15 not due to damper 13, such as from a guide, bearing, or from seals in end closure 32. Because the coefficient of static friction is in general greater than the coefficient of dynamic friction, it may be important that there be a brief force spike at the beginning of the delivery in order to start the motion of stopper 18. This pressure spike may also be required to insert needle 20 into the target injection site. This pressure spike can be created by a gap between plunger rod 15 and stopper 18.

In some embodiments, due to the variability of fill volume, the stopper position is somewhat unpredictable. It cannot be placed such that there is no head space, and when the syringe is installed in the autoinjector there must be a gap between the plunger rod and the stopper-primarily to ensure that there is no fluid displacement. This means that there is a gap between the plunger rod and the stopper.

At this point it is known what force plunger rod 15 must exert on stopper 18, and thus the configuration of damper 13 and energy source 17. In a preferred embodiment injector 11 is a platform system intended to be used for many formulations, and the energy source 17 is chosen from a mechanical spring, a 2-phase system, a combustion system or a reactive chemical system, and F_{source}, the force from energy source 17, is essentially fixed due to the complexity of making changes. In other cases, such as a compressed gas, the force from the energy source can be adjusted, for example by simply changing the pressure or fill amount of the pressurized gas source. In either case, damper 13 can be designed using the following equations:

$${F}_{damper} = \frac{μ A {v}_{plunger}}{{l}_{gap}} = {F}_{source} - {F}_{friction} - {F}_{stopper} - {F}_{pressure} \\ {F}_{damper} = \frac{μ A {v}_{0}}{{l}_{gap}} = {F}_{source} - {F}_{friction}$$

These two equalities can be met by adjusting two free parameters selected from the list including but not limited to l_{gap}, μ, and F_{source}. In some cases, F_{source}, F_{friction}, the mass of plunger rod 15, and the gap between plunger 15 and stopper 18 may be such that the system is insensitive to the amount of damping during the run in of plunger 15, and the damper may be designed solely based on the first equation, and only one free parameter may need to be adjusted. In some cases, the solution to the two equations above may not be achievable by reasonable adjustment of the viscosity, l_{gap}, or F_{source}, and the requirements on such things as needle 20 ID, the needle 20 length, the delivery time, the formulation volume, the formulation concentration, or the friction between stopper 18 and drug cartridge 19 will have to be loosened.

In one embodiment, parameters selected from the list including but not limited to viscosity, area of contact, l_{gap}, the gap between plunger rod 15 and stopper 18, and/or the force exerted by energy source are selected so that a maximum speed that the plunger rod 15 achieves prior to formulation being delivered from drug cartridge 19 is between about 0.1 m/s and about 100 m/s, preferably about 0.5 m/s to about 50 m/s, more preferably about 1 m/s to about 10 m/s. In one specific embodiment, the maximum speed that the plunger rod 15 achieves prior to formulation being delivered from drug cartridge 19 is about 0.1 m/s, about 0.5 m/s, about 1 m/s, about 1.5 m/s, about 2 m/s, about 2.5 m/s, about 3 m/s, about 3.5 m/s, about 4 m/s, about 4.5 m/s, about 5 m/s, about 5.5 m/s, about 6 m/s, about 6.5 m/s, about 7 m/s, about 7.5 m/s, about 8 m/s, about 8.5 m/s, about 9 m/s, about 9.5 m/s, about 10 m/s, about 11 m/s, about 12 m/s, about 13 m/s, about 14 m/s, about 15 m/s, about 16 m/s, about 17 m/s, about 18 m/s, about 19 m/s, about 20 m/s, about 25 m/s, about 30 m/s, about 35 m/s, about 40 m/s, about 45 m/s, about 50 m/s, about 55 m/s, about 60 m/s, about 65 m/s, about 70 m/s, about 75 m/s, about 80 m/s, about 85 m/s, about 90 m/s, about 95 m/s, or about 100 m/s.

In this or other embodiments, the viscosity, area of contact, l_{gap}, and/or the force exerted by the energy source are selected so that the average speed of plunger rod 15 after contacting stopper 18 is between about 0.5 mm/s to about 10 mm/s, preferably about 1 mm/s to about 7 mm/s, more preferably about 3 mm/s to about 4 mm/s.

In one embodiment, the average speed of plunger rod 15 after contacting stopper 18 is about 0.5 mm/s, about 1 mm/s, about 1.5 mm/s, about 2 mm/s, about 2.5 mm/s, about 3 mm/s, about 3.5 mm/s, about 4 mm/s, about 4.5 mm/s, about 5 mm/s, about 5.5 mm/s, about 6 mm/s, about 6.5 mm/s, about 7 mm/s, about 7.5 mm/s, about 8 mm/s, about 8.5 mm/s, about 9 mm/s, about 9.5 mm/s, about 10 mm/s, about 20 mm/s, or about 30 mm/s.

It can be seen from the equation above for F_{damper} that once the stopper, pressure, and source forces are determined, the equality can be held by adjusting the viscosity, area of contact A, or the gap l_{gap}. In the case of a platform system, A and/or l_{gap} may be fixed, in which case the viscosity may be used to optimize the system. In the case that the viscosity of damping medium 24 cannot be adjusted sufficiently, for example if damping medium 24 is not commercially available in the desired viscosity, then l_{gap} can be adjusted, for example by using insert 25.

The gap l_{gap} between the outside of the damped plunger rod 15 and the surface 23 is generally less than about 10 millimeters, preferably less than about 5 millimeters, more preferably less than about 2 millimeters, most preferably less than about 1 mm. In a particularly preferred embodiment, gap is less than or about 0.5 millimeter. In one specific embodiment, l_{gap} is about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, 4.1 mm, about 4.2 mm, about 4.3 mm, about 4.4 mm, about 4.5 mm, about 4.6 mm, about 4.7 mm, about 4.8 mm, about 4.9 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, or about 10.0 mm.

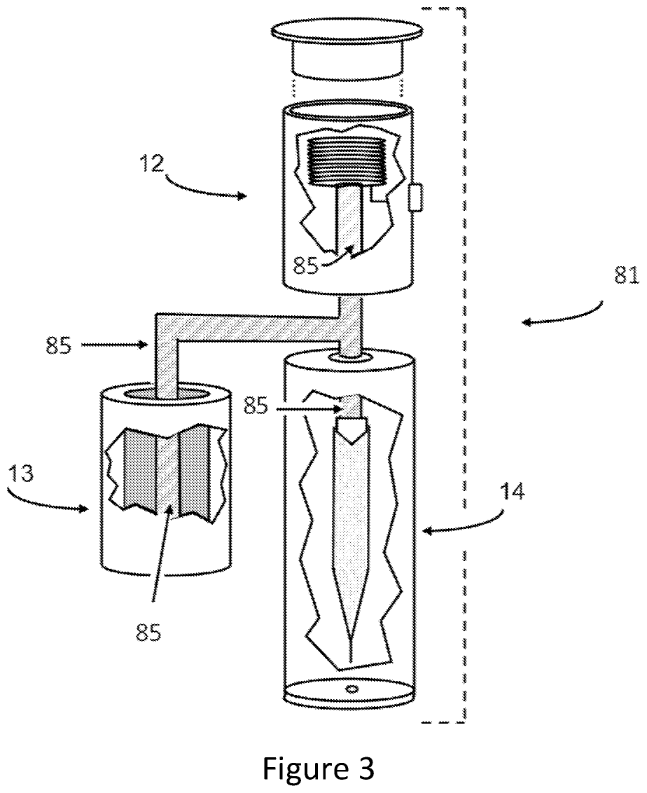

FIG. 3 show another embodiment of the current invention, which for example may be used if minimizing the length in autoinjector 81 is desired. Injector 81 is similar in many ways to Injector 11 as shown in FIG. 1, including energy source mechanism 12 and delivery section 14, which are conceptually the same as described above. Plunger rod 15 has been replaced with dual plunger rod 85. Dual plunger rod 85 enables the separation of the functions of damper 13 and delivery section 14. Damping mechanism 13 can now be in a dash pot configuration, wherein the end of the side portion of dual plunging rod 85 inside of damping medium 24 and surrounded by damping medium 24. However, this requires some airspace within damping mechanism 13 to receive displaced damping medium 24. In the embodiment where autoinjector 81 is a multi-dose autoinjector, there may be some evolution of the damping caused by repeatedly displacing damping medium 24 into the airspace. Thus, for multi-dose embodiments of autoinjector it is preferred that plunger rod pass through damping mechanism 13, and its design and function are essentially as described above for autoinjector 11.

### D. Properties of the Formulation

In one embodiment, the design of autoinjector 11 proceeds as follows. In general, the properties of the formulation are selected first based on the needs of the therapy, and are not free parameters.

In one embodiment, the injected formulation is a liquid, a liquid solution, a liquid suspension, a semi-solid, a gel, a hydrogel, a solid, a gas, a vapor, or a powder.

In another embedment, the dose of the formulation is selected based on the required therapy. In yet another embodiment, the dose of the formulation is less than about 200 mg, less than about 150 mg, less than about 100 mg, less than about 50, less than about 10 mg, less than about 5 mg, or less than about 1 mg. In one specific embodiment, the dose of the formulation is about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 35 mg, about 40 mg, about 45 mg, about 50 mg, about 55 mg, about 60 mg, about 70 mg, about 80 mg, about 90 mg, about 100 mg, about 120 mg, about 140 mg, about 160 mg, about 180 mg, or about 200 mg.

In one embodiment, the concentration of active ingredient in the formulation is less than 500 mg/ml, less than 100 mg/ml, less than 50 mg/ml, or less than 20 mg/ml. In another embodiment, the concentration of the active ingredient in the formulation is about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, or about 200 mg/ml.

In another embodiment, the dose volume is from about 0.05 ml to about 100 ml, from about 0.05 to about 10 ml, from about 1 ml to about 3 ml, from about 1 ml to about 5 ml, from about 1 ml to about 8 ml, from about 3 ml to about 5 ml, from about 3 ml to about 7 ml, from about 3 ml to about 9 ml, from about 5 ml to about 7 ml, from about 5 ml to about 9 ml, or from about 7 ml to about 9 ml. In yet another embodiment, the dose volume is about 0.05 ml, about 1 ml, about 1.5 ml, about 2 ml, about 2.5 ml, about 3 ml, about 3.5 ml, about 4 ml, about 4.5 ml, about 5 ml, about 1 ml, about 1.5 ml, about 2 ml, about 2.5 ml about 3 ml, about 3.5 ml, about 4 ml, about 4.5 ml, about 5 ml, about 5.5 ml, about 6 ml, about 6.5 ml, about 7 ml, about 7.5 ml, about 8 ml, about 8.5 ml, about 9 ml, about 9.5 ml, or about 10 ml.

In one embodiment, the formulation viscosity may be between about 1 cP and about 200 cP, between about 10 cP and about 100 cP, or between about 20 cP and about 50 cP. In another embodiment, the formulation viscosity is less than about 200 cP, less than about 100 cP, less than about 50 cP, or less than about 20 cP. In yet another embodiment, the formulation viscosity is about 5 cP, about 10 cP, about 20 cP, about 30 cP, about 40 cP, about 50 cP, about 60 cP, about 70 cP, about 80 cP, about 90 cP, about 100 cP, about 110 cP, about 120 cP, about 130 cP, about 140 cP, about 150 cP, about 160 cP, about 170 cP, about 180 cP, about 190 cP, about 200 cP, about 250 cP, about 300 cP, about 350 cP, about 400 cP, about 450 cP, or about 500 cP.

In some instances, the formulation and dose requirements will call for more than one injection, each of which has properties as described above.

In some embodiments, autoinjector 11 is designed to inject a liquid formulation through hypodermic needle 20. In this embodiment, the dose of the active ingredient is selected based on the required therapy. Generally, the dose is less than about 100 mg, preferably less than about 10 mg, more preferably the dose is less than about 5 mg, most preferably the dose is less than or about 1 mg. If the dose is high, the concentration of the active ingredient in the liquid formulation is then determined by, for example, the solubility of the formulation, in order to minimize the required injected volume. Generally, the concentration of active ingredient is less than or about 500 mg/ml, preferably less than or about 100 mg/ml, more preferably less than about 10 mg/ml, still more preferably less than or about 1 mg/ml, most preferably less than or about 0.1 mg/ml. Once the concentration and dose are determined, the dose volume can be determined by

volume=dose/concentration

Preferably the dose volume is in the range of about 0.05 to about 10 ml, more preferably in the range of about 0.05 to about 5 ml, most preferably is the range of about 0.1 to about 1 ml. In some instances, the formulation and dose requirements will call for more than one injection, each of which has properties as described above. For needle 20, it is generally desirable to have the smallest diameter (highest needle gauge) to minimize pain and needle phobia.

Drug cartridge 19 of the device may be filled with colony stimulating factors, such as granulocyte colony stimulating factor (G-CSF). Such G-CSF agents include, but are not limited to, Neupogen® (filgrastim) and Neulasta® (pegfilgrastim). In various other embodiments, the drug delivery device may be used with various pharmaceutical products, such as an erythropoiesis stimulating agent (ESA), which may be in a liquid or a lyophilized form. An ESA is any molecule that stimulates erythropoiesis, such as Epogen® (epoetin alfa), Aranesp® (darbepoetin alfa), Dynepo® (epoetin delta), Mircera® (methyoxy polyethylene glycolepoetin beta), HematideR, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, as well as the molecules or variants or analogs thereof as disclosed in the following patents or patent applications, each of which is herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,986,047; 6,583,272; 7,084,245; and 7,271,689; and PCT Publication Nos. WO 91/05867; WO 95/05465; WO 96/40772; WO 00/24893; WO 01/81405; and WO 2007/136752.

An ESA can be an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, epoetin alfa, epoetin beta, epoetin delta, epoetin omega, epoetin iota, epoetin zeta, and analogs thereof, pegylated erythropoietin, carbamylated erythropoietin, mimetic peptides (including EMPlhematide), and mimetic antibodies. Exemplary erythropoiesis stimulating proteins include erythropoietin, darbepoetin, erythropoietin agonist variants, and peptides or antibodies that bind and activate erythropoietin receptor (and include compounds reported in U.S. Publication Nos. 2003/0215444 and 2006/0040858, the disclosures of each of which is incorporated herein by reference in its entirety) as well as erythropoietin molecules or variants or analogs thereof as disclosed in the following patents or patent applications, which are each herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,830,851; 5,856,298; 5,986,047; 6,030,086; 6,310,078; 6,391,633; 6,583,272; 6,586,398; 6,900,292; 6,750,369; 7,030,226; 7,084,245; and 7,217,689; U.S. Publication Nos. 2002/0155998; 2003/0077753; 2003/0082749; 2003/0143202; 2004/0009902; 2004/0071694; 2004/0091961; 2004/0143857; 2004/0157293; 2004/0175379; 2004/0175824; 2004/0229318; 2004/0248815; 2004/0266690; 2005/0019914; 2005/0026834; 2005/0096461; 2005/0107297; 2005/0107591; 2005/0124045; 2005/0124564; 2005/0137329; 2005/0142642; 2005/0143292; 2005/0153879; 2005/0158822; 2005/0158832; 2005/0170457; 2005/0181359; 2005/0181482; 2005/0192211; 2005/0202538; 2005/0227289; 2005/0244409; 2006/0088906; and 2006/0111279; and PCT Publication Nos. WO 91/05867; WO 95/05465; WO 99/66054; WO 00/24893; WO 01/81405; WO 00/61637; WO 01/36489; WO 02/014356; WO 02119963; WO 02/20034; WO 02/49673; WO 02/085940; WO 031029291; WO 2003/055526; WO 2003/084477; WO 2003/094858; WO 2004/002417; WO 2004/002424; WO 2004/009627; WO 2004/024761; WO 2004/033651; WO 2004/035603; WO 2004/043382; WO 2004/101600; WO 2004/101606; WO 20041/01611; WO 2004/106373; WO 2004/018667; WO 2005/001025; WO 2005/001136; WO 2005/021579; WO 2005/025606; WO 2005/032460; WO 2005/051327; WO 2005/063808; WO 2005/063809; WO 2005/070451; WO 2005/081687; WO 2005/084711; WO 2005/103076; WO 2005/100403; WO 2005/092369; WO 2006/50959; WO 2006/02646; and WO 2006/29094.

Examples of other pharmaceutical products for use with the device may include, but are not limited to, antibodies such as Vectibix® (panitumumab), Xgeva™ (denosumab) and Prolia™ (denosamab); other biological agents such as Enbrel® (etanercept, TNF-receptor/Fc fusion protein, TNF blocker), Neulasta® (pegfilgrastim, pegylated filgastrim, pegylated G-CSF, pegylated hu-Met-G-CSF), Neupogen® (filgrastim, G-CSF, hu-MetG-CSF), and Nplate® (romiplostim); small molecule drugs such as Sensipar® (cinacalcet). The device may also be used with a therapeutic antibody, a polypeptide, a protein or other chemical, such as an iron, for example, ferumoxytol, iron dextrans, ferric glyconate, and iron sucrose. The pharmaceutical product may be in liquid form, or reconstituted from lyophilized form.

Among particular illustrative proteins are the specific proteins set forth below, including fusions, fragments, analogs, variants or derivatives thereof.

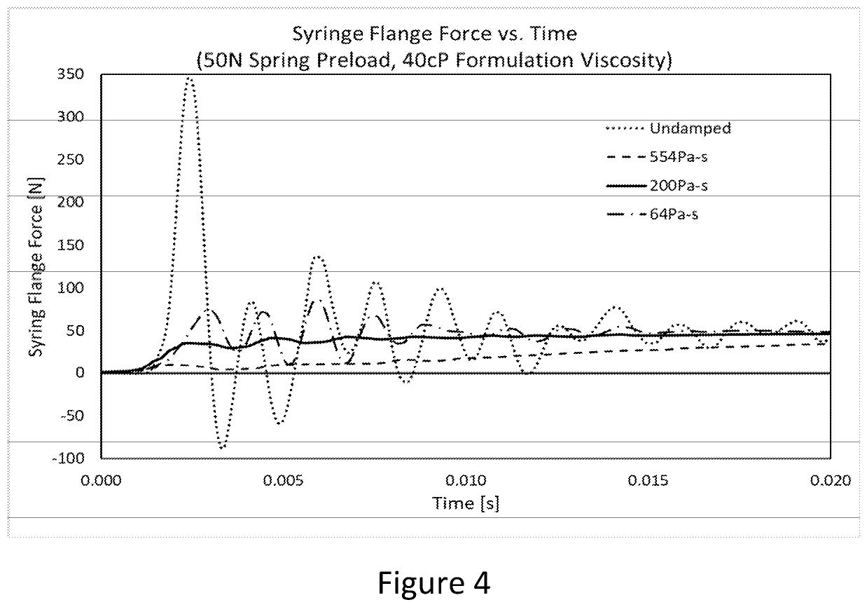

OPGL specific antibodies, peptibodies, and related proteins, and the like (also referred to as RANKL specific antibodies, peptibodies and the like), including fully humanized and human OPGL specific antibodies, particularly fully humanized monoclonal antibodies, including but not limited to the antibodies described in PCT Publication No. WO 03/002713, which is incorporated herein in its entirety as to OPGL specific antibodies and antibody related proteins, particularly those having the sequences set forth therein, particularly, but not limited to, those denoted therein: 9I17; 18B2; 2D8; 2E11; 16E1; a nd 22B3, including the OPGL specific antibodies having either the light chain of sequence identification number: 2 as set forth therein in FIG. 2 and/or the heavy chain of sequence identification number: 4, as set forth therein in FIG. 4, each of which is individually and specifically incorporated by reference herein in its entirety fully as disclosed in the foregoing publication;

Myostatin binding proteins, peptibodies, and related proteins, and the like, including myostatin specific peptibodies, particularly those described in U.S. Publication No. 2004/0181033 and PCT Publication No. WO 2004/058988, which are incorporated by reference herein in their entirety particularly in parts pertinent to myostatin specific peptibodies, including but not limited to peptibodies of the mTN8-19 family, including those of sequence identification numbers: 305-351, including TN8-19-1 through TN8-19-40, TN8-19 con1 and TN8-19 con2; peptibodies of the mL2 family of sequence identification numbers: 357-383; the mL15 family of sequence identification numbers: 384-409; the mL17 family of sequence identification numbers: 410-438; the mL20 family of sequence identification numbers: 439-446; the mL21 family of sequence identification numbers: 447-452: the mL24 family of sequence identification numbers: 453-454; and those of sequence identification numbers: 615-631, each of which is individually and specifically incorporated by reference herein in their entirety fully as disclosed in the foregoing publication;

IL-4 receptor specific antibodies, peptibodies, and related proteins, and the like, particularly those that inhibit activities mediated by binding of IL-4 and/or IL-13 to the receptor, including those described in PCT Publication No. WO 2005/047331 or PCT Application No. PCT/US2004/37242 and in U.S. Publication No. 2005/112694, which are incorporated herein by reference in their entirety particularly in parts pertinent to IL-4 receptor specific antibodies, particularly such antibodies as are described therein, particularly, and without limitation, those designated therein: LIH1; LIH2; LIH3; LIH4; LIH5; LIH6; LIH7; LIH8; LIH9; LIH10; LIH11; L2H1; L2H2; L2H3; L2H4; L2H5; L2H6; L2H7; L2H8; L2H9; L2H10; L2H11; L2H12; L2H13; L2H14; L3H1; L4H1; L5H1; L6H1, each of which is individually and specifically incorporated by reference herein in its entirety fully as disclosed in the foregoing publication;

Interleukin I-receptor 1 (“IL1-R1”) specific antibodies, peptibodies, and related proteins, and the like, including but not limited to those described in U.S. Publication No. 2004/097712, which is incorporated herein by reference in its entirety in parts pertinent to ILI-RI specific binding proteins, monoclonal antibodies in particular, especially, without limitation, those designated therein: 15CA, 26F5, 27F2, 24E12, and 10H7, each of which is individually and specifically incorporated by reference herein in its entirety fully as disclosed in the aforementioned publication;

Ang2 specific antibodies, peptibodies, and related proteins, and the like, including but not limited to those described in PCT Publication No. WO 03/057134 and U.S. Publication No. 2003/0229023, each of which is incorporated herein by reference in its entirety particularly in parts pertinent to Ang2 specific antibodies and peptibodies and the like, especially those of sequences described therein and including but not limited to: L1(N): L1(N) WT; L1(N) 1K WT; 2xL1(N); 2xL1(N) WT; Con4(N), Con4(N) 1K WT, 2xCon4(N) 1K; L1C; L1C 1K; 2xL1C; Con4C; Con4C 1K; 2xCon4C 1K; Con4-L1 (N); Con4-L1C; TN-12-9(N); C17(N); TN8-8(N); TN8-14(N); Con 1(N), also including antiAng2 antibodies and formulations such as those described in PCT Publication No. WO 2003/030833 which is incorporated herein by reference in its entirety as to the same, particularly Ab526; Ab528; Ab531; Ab533; Ab535; Ab536; Ab537; Ab540; Ab543; Ab544; Ab545; Ab546; A551; Ab553; Ab555; Ab558; Ab559; Ab565; AbF1AbFD; AbFE; AbFJ; AbFK; AbG1D4; AbGC1E8; AbH1C12; Ab1A1; Ab1F; Ab1K, Ab1P; and Ab1P, in their various permutations as described therein, each of which is individually and specifically incorporated by reference herein in its entirety fully as disclosed in the foregoing publication;

NGF specific antibodies, peptibodies, and related proteins, and the like including, in particular, but not limited to those described in U.S. Publication No. 2005/0074821 and U.S. Pat. No. 6,919,426, which are incorporated herein by reference in their entirety particularly as to NGF-specific antibodies and related proteins in this regard, including in particular, but not limited to, the NGF-specific antibodies therein designated 4D4, 4G6, 6H9, 7H2, 14D10 and 14D11, each of which is individually and specifically incorporated by reference herein in its entirety fully as disclosed in the foregoing publication;

CD22 specific antibodies, peptibodies, and related proteins, and the like, such as those described in U.S. Pat. No. 5,789,554, which is incorporated herein by reference in its entirety as to CD22 specific antibodies and related proteins, particularly human CD22 specific antibodies, such as but not limited to humanized and fully human antibodies, including but not limited to humanized and fully human monoclonal antibodies, particularly including but not limited to human CD22 specific IgG antibodies, such as, for instance, a dimer of a human-mouse monoclonal hLL2 gamma-chain disulfide linked to a human-mouse monoclonal hLL2 kappa-chain, including, but limited to, for example, the human CD22 specific fully humanized antibody in Epratuzumab, CAS registry number 501423-23-0;

IGF-1 receptor specific antibodies, peptibodies, and related proteins, and the like, such as those described in PCT Publication No. WO 06/069202, which is incorporated herein by reference in its entirety as to IGF-1 receptor specific antibodies and related proteins, including but not limited to the IGF-1 specific antibodies therein designated LIH1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47 H47, L48H48, L49H49, L50H50, L51H51, L52H52, and IGF-IR-binding fragments and derivatives thereof, each of which is individually and specifically incorporated by reference herein in its entirety fully as disclosed in the foregoing publication;

Also among non-limiting examples of anti-IGF-IR antibodies for use in the methods and compositions of the present invention are each and all of those described in:
(i) U.S. Publication No. 2006/0040358 (published Feb. 23, 2006), 2005/0008642 (published Jan. 13, 2005), 2004/0228859 (published Nov. 18, 2004), including but not limited to, for instance, antibody 1A (DSMZ Deposit No. DSM ACC 2586), antibody 8 (DSMZ Deposit No. DSM ACC 2589), antibody 23 (DSMZ Deposit No. DSM ACC2588) and antibody 18 as described therein;
(ii) PCT Publication No. WO 06/138729 (published Dec. 28, 2006) and WO 05/016970 (published Feb. 24, 2005), and Lu et al. (2004), J. Biol. Chem. 279:2856-2865, including but not limited to antibodies 2F8, A12, and IMC-A12 as described therein;
(iii) PCT Publication No. WO 07/012614 (published Feb. 1, 2007), WO 07/000328 (published Jan. 4, 2007), WO 06/013472 (published Feb. 9, 2006), WO 05/058967 (published Jun. 30, 2005), and WO 03/059951 (published Jul. 24, 2003);
(iv) U.S. Publication No. 2005/0084906 (published Apr. 21, 2005), including but not limited to antibody 7C10, chimaeric antibody C7C10, antibody h7C10, antibody 7H2M, chimaeric antibody *7C10, antibody GM 607, humanized antibody 7C10 version 1, humanized antibody 7C10 version 2, humanized antibody 7C10 version 3, and antibody 7H2HM, as described therein;
(v) U.S. Publication Nos. 2005/0249728 (published Nov. 10, 2005), 2005/0186203 (published Aug. 25, 2005), 2004/0265307 (published Dec. 30, 2004), and 2003/0235582 (published Dec. 25, 2003) and Maloney et al. (2003), Cancer Res. 63:5073-5083, including but not limited to antibody EM164, resurfaced EM164, humanized EM164, huEM164 v 1.0, huEM164 v 1.1, huEM164 v 1.2, and huEM164 v 1.3 as described therein;
(vi) U.S. Pat. No. 7,037,498 (issued May 2, 2006), U.S. Publication Nos. 2005/0244408 (published Nov. 30, 2005) and 2004/0086503 (published May 6, 2004), and Cohen, et al. (2005), Clinical Cancer Res. 11:2063-2073, e.g., antibody CP-751,871, including but not limited to each of the antibodies produced by the hybridomas having the ATCC accession numbers PTA-2792, PTA-2788, PTA-2790, PTA-2791, PTA-2789, PTA-2793, and antibodies 2.12.1, 2.13.2, 2.14.3, 3.1.1, 4.9.2, and 4.17.3, as described therein;
(vii) U.S. Publication No. 2005/0136063 (published Jun. 23, 2005) and 2004/0018191 (published Jan. 29, 2004), including but not limited to antibody 19D12 and an antibody comprising a heavy chain encoded by a polynucleotide in plasmid 15H12/19D12 HCA (y4), deposited at the ATCC under number PTA-5214, and a light chain encoded by a polynucleotide in plasmid 15H12/19D12 LCFC(K), deposited at the ATCC under number PTA-5220, as described therein; and
(viii) U.S. Publication No. 2004/0202655 (published Oct. 14, 2004), including but not limited to antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, as described therein; each and all of which are herein incorporated by reference in their entireties, particularly as to the aforementioned antibodies, peptibodies, and related proteins and the like that target IGF-1 receptors;

B-7 related protein 1 specific antibodies, peptibodies, related proteins and the like (“B7RP-1,” also is referred to in the literature as B7H2, ICOSL, B7h, and CD275), particularly B7RP-specific fully human monoclonal IgG2 antibodies, particularly fully human IgG2 monoclonal antibody that binds an epitope in the first immunoglobulin-like domain of B7RP-1, especially those that inhibit the interaction of B7RP-1 with its natural receptor, ICOS, on activated T cells in particular, especially, in all of the foregoing regards, those disclosed in U.S. Publication No. 2008/0166352 and PCT Publication No. WO 07/011941, which are incorporated herein by reference in their entireties as to such antibodies and related proteins, including but not limited to antibodies designated therein as follow: 16H (having light chain variable and heavy chain variable sequences sequence identification number: 1 and sequence identification number: 7 respectively therein): 5D (having light chain variable and heavy chain variable sequences sequence identification number: 2 and sequence identification number: 9 respectively therein): 2H (having light chain variable and heavy chain variable sequences sequence identification number: 3 and sequence identification number: 10 respectively therein): 43H (having light chain variable and heavy chain variable sequences sequence identification number: 6 and sequence identification number: 14 respectively therein): 41H (having light chain variable and heavy chain variable sequences sequence identification number: 5 and sequence identification number: 13 respectively therein); and 15H (having light chain variable and heavy chain variable sequences sequence identification number: 4 and sequence identification number: 12 respectively therein), each of which is individually and specifically incorporated by reference herein in its entirety fully as disclosed in the foregoing publication;

IL-15 specific antibodies, peptibodies, and related proteins, and the like, such as, in particular, humanized monoclonal antibodies, particularly antibodies such as those disclosed in U.S. Publication Nos. 2003/0138421; 2003/023586; and 2004/0071702; and U.S. Pat. No. 7,153,507, each of which is incorporated herein by reference in its entirety as to IL-15 specific antibodies and related proteins, including peptibodies, including particularly, for instance, but not limited to, HuMax IL-15 antibodies and related proteins, such as, for instance, 146B7:

IFN gamma specific antibodies, peptibodies, and related proteins and the like, especially human IFN gamma specific antibodies, particularly fully human anti-IFN gamma antibodies, such as, for instance, those described in U.S. Publication No. 2005/0004353, which is incorporated herein by reference in its entirety as to IFN gamma specific antibodies, particularly, for example, the antibodies therein designated 1118; 1118*; 1119; 1121; and 1121*. The entire sequences of the heavy and light chains of each of these antibodies, as well as the sequences of their heavy and light chain variable regions and complementarity determining regions, are each individually and specifically incorporated by reference herein in its entirety fully as disclosed in the foregoing publication and in Thakur et al. (1999), Mol. Immunol. 36:1107-1115. In addition, description of the properties of these antibodies provided in the foregoing publication is also incorporated by reference herein in its entirety. Specific antibodies include those having the heavy chain of sequence identification number: 17 and the light chain of sequence identification number: 18; those having the heavy chain variable region of sequence identification number: 6 and the light chain variable region of sequence identification number: 8; those having the heavy chain of sequence identification number: 19 and the light chain of sequence identification number: 20; those having the heavy chain variable region of sequence identification number: 10 and the light chain variable region of sequence identification number: 12; those having the heavy chain of sequence identification number: 32 and the light chain of sequence identification number: 20; those having the heavy chain variable region of sequence identification number: 30 and the light chain variable region of sequence identification number: 12; those having the heavy chain sequence of sequence identification number: 21 and the light chain sequence of sequence identification number: 22; those having the heavy chain variable region of sequence identification number 14 and the light chain variable region of sequence identification number: 16: those having the heavy chain of sequence identification number: 21 and the light chain of sequence identification number: 33; and those having the heavy chain variable region of sequence identification number: 14 and the light chain variable region of sequence identification number: 31, as disclosed in the foregoing publication. A specific antibody contemplated is antibody 1119 as disclosed in the foregoing U.S. publication and having a complete heavy chain of sequence identification number: 17 as disclosed therein and having a complete light chain of sequence identification number: 18 as disclosed therein:

TALL-1 specific antibodies, peptibodies, and the related proteins, and the like, and other TALL specific binding proteins, such as those described in U.S. Publication Nos. 2003/0195156 and 2006/0135431, each of which is incorporated herein by reference in its entirety as to TALL-1 binding proteins, particularly the molecules of Tables 4 and 5B, each of which is individually and specifically incorporated by reference herein in its entirety fully as disclosed in the foregoing publications:

Parathyroid hormone (“PTH”) specific antibodies, peptibodies, and related proteins, and the like, such as those described in U.S. Pat. No. 6,756,480, which is incorporated herein by reference in its entirety, particularly in parts pertinent to proteins that bind PTH:

Thrombopoietin receptor (“TPO-R”) specific antibodies, peptibodies, and related proteins, and the like, such as those described in U.S. Pat. No. 6,835,809, which is herein incorporated by reference in its entirety, particularly in parts pertinent to proteins that bind TPO-R:

Hepatocyte growth factor (“HGF”) specific antibodies, peptibodies, and related proteins, and the like, including those that target the HGF/SF:cMet axis (HGF/SF:c-Met), such as the fully human monoclonal antibodies that neutralize hepatocyte growth factor/scatter (HGF/SF) described in U.S. Publication No. 2005/0118643 and PCT Publication No. WO 2005/017107, huL2G7 described in U.S. Pat. No. 7,220,410 and OA-5d5 described in U.S. Pat. Nos. 5,686,292 and 6,468,529 and in PCT Publication No. WO 96/38557, each of which is incorporated herein by reference in its entirety, particularly in parts pertinent to proteins that bind HGF;

TRAIL-R2 specific antibodies, peptibodies, related proteins and the like, such as those described in U.S. Pat. No. 7,521,048, which is herein incorporated by reference in its entirety, particularly in parts pertinent to proteins that bind TRAIL-R2;

Activin A specific antibodies, peptibodies, related proteins, and the like, including but not limited to those described in U.S. Publication No. 2009/0234106, which is herein incorporated by reference in its entirety, particularly in parts pertinent to proteins that bind Activin A;

TGF-beta specific antibodies, peptibodies, related proteins, and the like, including but not limited to those described in U.S. Pat. No. 6,803,453 and U.S. Publication No. 2007/0110747, each of which is herein incorporated by reference in its entirety, particularly in parts pertinent to proteins that bind TGF-beta;

Amyloid-beta protein specific antibodies, peptibodies, related proteins, and the like, including but not limited to those described in PCT Publication No. WO 2006/081171, which is herein incorporated by reference in its entirety, particularly in parts pertinent to proteins that bind amyloid-beta proteins. One antibody contemplated is an antibody having a heavy chain variable region comprising sequence identification number: 8 and a light chain variable region having sequence identification number: 6 as disclosed in the foregoing publication;

c-Kit specific antibodies, peptibodies, related proteins, and the like, including but not limited to those described in U.S. Publication No. 2007/0253951, which is incorporated herein by reference in its entirety, particularly in parts pertinent to proteins that bind c-Kit and/or other stem cell factor receptors;

OX40L specific antibodies, peptibodies, related proteins, and the like, including but not limited to those described in U.S. Publication No. 2006/0002929, which is incorporated herein by reference in its entirety, particularly in parts pertinent to proteins that bind OX40L and/or other ligands of the OX40 receptor; and

Other exemplary proteins, including Activase® (alteplase, tPA); Aranesp® (darbepoetin alfa): Epogen® (epoetin alfa, or erythropoietin); GLP-1, Avonex® (interferon beta-1a); Bexxar® (tositumomab, anti-CD22 monoclonal antibody); Betaseron® (interferon-beta): Campath® (alemtuzumab, anti-CD52 monoclonal antibody); Dynepo® (epoetin delta); Velcade® (bortezomib); MLN0002 (antia4ß7 mAb); MLN1202 (anti-CCR2 chemokine receptor mAb): Enbrel® (etanercept, TNF-receptor/Fc fusion protein, TNF blocker): Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta): Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab): TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin human interleukin-11); Neulasta® (pegylated filgastrim, pegylated G-CSF, pegylated hu-Met-G-CSF); Neupogen® (filgrastim, G-CSF, hu-MetG-CSF); Orthoclone OKT3® (muromonab-CD3, anti-CD3 monoclonal antibody); Procrit® (epoetin alfa): Remicade® (infliximab, anti-TNFα monoclonal antibody); Reopro® (abciximab, anti-GP IIb/Ilia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, antia4integrin mAb); Valortim® (MDX-1303, anti-B. anthracis protective antigen mAb); ABthrax™; Vectibix® (panitumumab); Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, antia5ß1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); antiCD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; antiCTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; antiganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-1103); anti-IGFIR mAb; anti-IGF-IR mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); anti-LLY antibody; BMS-66513; anti-Mannose Receptor/hCGB mAb (MDX-1307); anti-mesothelin dsFv-PE38 conjugate (CAT-500); anti-PD1 mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; antiVEGFR/Flt-1 mAb; anti-ZP3 mAb (HuMax-ZP3): NVS Antibody #1; and NVS Antibody #2.

Also included can be a sclerostin antibody, such as but not limited to romosozumab, blosozumab, or BPS 804 (Novartis). Further included can be therapeutics such as rilotumumab, bixalomer, trebananib, ganitumab, conatumumab motesanib diphosphate, brodalumab, vidupiprant, panitumumab, denosumab, NPLATE, PROLIA, VECTIBIX or XGEVA. Additionally, included in the device can be a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab), as well as molecules, variants, analogs or derivatives thereof as disclosed in the following patents or patent applications, each of which is herein incorporated by reference in its entirety for all purposes: U.S. Pat. No. 8,030,547, U.S. Publication No. 2013/0064825, WO2008/057457, WO2008/057458, WO2008/057459, WO2008/063382, WO2008/133647, WO2009/100297, WO2009/100318, WO2011/037791, WO2011/053759, WO2011/053783, WO2008/125623, WO2011/072263, WO2009/055783, WO2012/0544438, WO2010/029513, WO2011/111007, WO2010/077854, WO2012/088313, WO2012/101251, WO2012/101252, WO2012/101253, WO2012/109530, and WO2001/031007.

Also included can be talimogene laherparepvec or another oncolytic HSV for the treatment of melanoma or other cancers. Examples of oncolytic HSV include, but are not limited to talimogene laherparepvec (U.S. Pat. Nos. 7,223,593 and 7,537,924); OncoVEXGALV/CD (U.S. Pat. No. 7,981,669): OrienXOIO (Lei et al. (2013), World J. Gastroenterol., 19:5138-5143); G207, 1716; NV1020; NV12023; NV1034 and NV1042 (Vargehes et al. (2002), Cancer Gene Ther., 9(12):967-978). Also included are TIMPs. TIMPs are endogenous tissue inhibitors of metalloproteinases (TIMPs) and are important in many natural processes. TIMP-3 is expressed by various cells or and is present in the extracellular matrix: it inhibits all the major cartilage-degrading metalloproteases, and may play a role in role in many degradative diseases of connective tissue, including rheumatoid arthritis and osteoarthritis, as well as in cancer and cardiovascular conditions. The amino acid sequence of TIMP-3, and the nucleic acid sequence of a DNA that encodes TIMP-3, are disclosed in U.S. Pat. No. 6,562,596, issued May 13, 2003, the disclosure of which is incorporated by reference herein. Description of TIMP mutations can be found in U.S. Publication No. 2014/0274874 and PCT Publication No. WO 2014/152012.

Also included are antagonistic antibodies for human calcitonin gene-related peptide (CGRP) receptor and bispecific antibody molecule that target the CGRP receptor and other headache targets. Further information concerning these molecules can be found in PCT Application No. WO 2010/075238.

Additionally, bispecific T cell engager (BiTER) antibodies, e.g. BLINCYTOR (blinatumomab), can be used in the device. Alternatively, included can be an APJ large molecule agonist e.g., apelin or analogues thereof in the device. Information relating to such molecules can be found in PCT Publication No. WO 2014/099984.

In certain embodiments, the medicament comprises a therapeutically effective amount of an anti-thymic stromal lymphopoietin (TSLP) or TSLP receptor antibody. Examples of anti-TSLP antibodies that may be used in such embodiments include, but are not limited to, those described in U.S. Pat. Nos. 7,982,016, and 8,232,372, and U.S. Publication No. 2009/0186022. Examples of anti-TSLP receptor antibodies include, but are not limited to, those described in U.S. Pat. No. 8,101,182. In particularly preferred embodiments, the medicament comprises a therapeutically effective amount of the anti-TSLP antibody designated as A5 within U.S. Pat. No. 7,982,016.

### E. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1—Evaluating Injection Performance Using Damping Greases with Different Viscosities

An apparatus was developed to test the embodiment of autoinjector 11 presented in FIG. 1 and described above. Delivery section 14 was a needle and syringe. Needle 20 was a 17 mm long 29 gauge hypodermic needle, and drug cartridge 19 was a 1 mL syringe and stopper (long version) per ISO11040-4:2015. A force transducer was placed below the syringe flange to determine the force when the plunger rod contacts and then drives forward the syringe stopper. Damping mechanism 13 was concentric right circular cylinders of length 19.05 mm, with a gap between cylinders of 0.835 mm. Injection performance utilizing damping greases with dynamic viscosities of 554 Pa-s (554,000 cP, Nye Lubricants PG 44A), 164 Pa-s (164,000 cP, Nyogel 767A), and 64 Pa-s (64,000 cP, Nyogel 774VH) was characterized and compared to performance without damping grease. A coil spring with a spring constant of 1017 N/m was used for the energy source.

As can be seen from FIG. 4 (data generated with a 50 N spring preload and 40 cP formulation viscosity), in the absence of damping grease (dotted line) there is a significant force spike of about 350 N when the plunger rod impacted the stopper, following by a period of oscillation or “ringing” where the force varies. The maximum force is about 7× the force required to deliver the formulation. The use of a damping medium with a viscosity of 64 Pa-s reduced the peak flange force to less than about 75 N, although significant ringing was still seen, implying the system was still underdamped. The use of a damping medium of 554 Pa-s resulted in a long slow approach to the equilibrium value, showing that the system with this damping medium was underdamped. Using a damping medium of 200 Pa-s, the force rapidly approached the equilibrium value with minimal ringing (“critically damped”), showing the system was nearly optimized.

### Example 2—Evaluating Injection Performance Using Different Spring Preloads

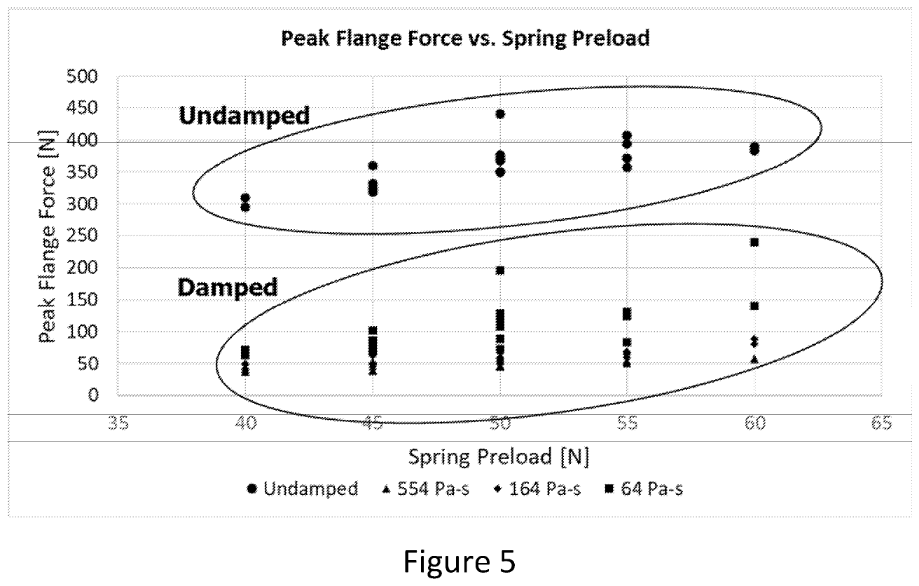

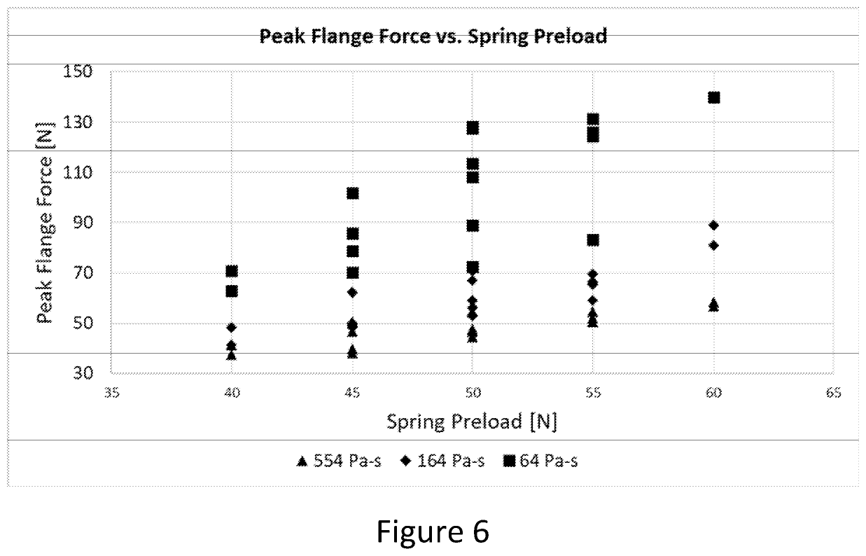

The experiment described in the previous paragraph was repeated with replicates at spring preloads of 40, 45, 50, 55, and 60 N, as shown in FIGS. 5 and 6. As can be seen in FIG. 5, damping using the system described above with any of the damping media results in a significantly reduced peak flange force as compared to no damping medium. As described previously, which damping medium to use depends on the actual implementation of the autoinjector, but by way of example, one might select a preload based on the desired delivery time, needle gauge and length, and formulation viscosity, and then select the 64 Pa-s damping medium for use with a 40 N preload, the 164 Pa-s damping medium for use with a 50 N preload, and the 554 Pa-s damping medium for use with a 60 N spring preload.

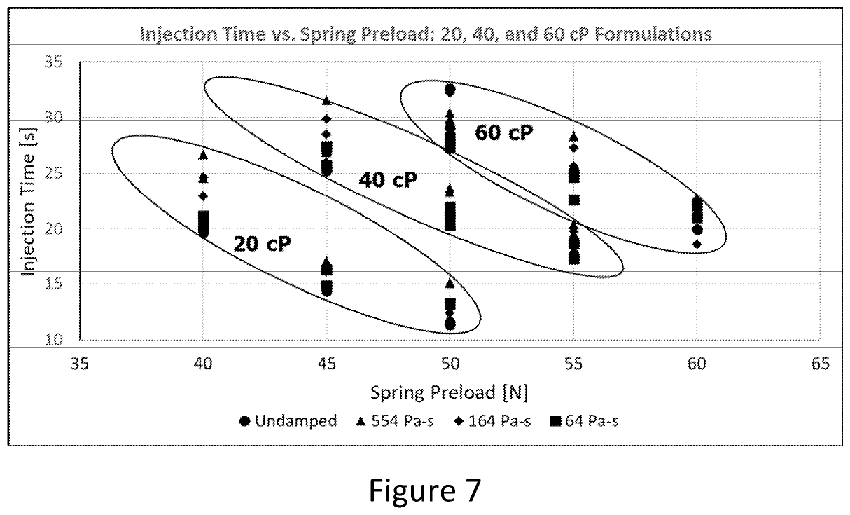

FIG. 7 shows the injection time vs. spring preload for various formulation viscosities, and damping media. By way of example, using a 29 gauge 17 mm long needle, if one desired a delivery time of approximately 18 seconds one might select a spring preload between about 40 N and about 45 N, for a 20 cP formulation, a spring preload of between about 50 N and about 60 N for a 40 cP formulation, and a spring preload of between about 60 N and about 70 N for a 60 cP formulation. The viscosity of the damping medium can then be selected to reduce the initial force spike as described above.

The instant invention is shown and described herein in a manner which is considered to be the most practical and preferred embodiments. It is recognized, however, that departures may be made therefrom which are within the scope of the invention and that obvious modifications will occur to one skilled in the art upon reading this disclosure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An autoinjector for injecting a formulation into a subject in need thereof, wherein the autoinjector comprises:
(a) a plunger rod comprising a first surface of the plunger rod,
(b) an energy source mechanism comprising an energy source and a trigger button,
(c) a damping mechanism comprising a second surface and a damping medium that is located between the first surface and the second surface, and
(d) a delivery section comprising a drug cartridge,
wherein upon applying a force on the trigger button, the trigger button triggers a release of energy from the energy source to a plunger rod which is configured to transmit more than about 10% of released energy to the drug cartridge and causes at least a portion of the formulation to be delivered from the drug cartridge to the subject.

2. The autoinjector of claim 1, wherein the first surface and the second surface are in contact with the damping medium and wherein after applying the force on the trigger button, a relative motion between the first surface and the second surface results in Couette flow that damps out force and pressure spikes during delivery of the formulation.

3. The autoinjector of claim 1, wherein the energy source comprises electrical energy, mechanical springs, or compressed gas.

4. The autoinjector of claim 1, wherein the damping medium is a Newtonian or non-Newtonian fluid or material.

5. The autoinjector of claim 4, wherein the damping medium has a viscosity of less than about 100,000,000 cP.

6. The autoinjector of claim 1, wherein the delivery section further comprises a distal end of a plunger rod, a stopper, a needle, an orifice, and a housing with a distal end.

7. The autoinjector of claim 1, wherein the formulation is administered intravenously, subcutaneously, intramuscularly, or intradermally.

8. The autoinjector of claim 1, wherein a force is exerted by the energy source in the range of about 10 N to about 200 N.

9. The autoinjector of claim 1, wherein more than about 20% of the released energy is absorbed by the damping medium.

10. The autoinjector of claim 1, wherein between about 10% and about 90% of the released energy is absorbed by the damping medium.

11. The autoinjector of claim 1, wherein the autoinjector is a single-dose disposable device, a multi-dose disposable device, or a multi-dose refillable device.

12. The autoinjector of claim 11, wherein the autoinjector is self-contained and portable.

13. The autoinjector of claim 12, wherein the autoinjector comprises a self-contained power source.

14. The autoinjector of claim 1, wherein the formulation is provided in liquid, liquid solution, liquid suspension, semi-solid, gel, hydrogel, solid, gas, vapor, or powder form.

15. The autoinjector of claim 14, wherein the formulation is a liquid formulation.

16. The autoinjector of claim 15, wherein the liquid formulation comprises a drug and a carrier and wherein the formulation has a viscosity of at least about 1 cP.

17. The autoinjector of claim 1, wherein the first surface and the second surface are each comprised of a section of a cylinder.

18. The autoinjector of claim 1, wherein the first surface and the second surface are each comprised of a section of a right circular cylinder, and wherein the right circular cylinders have axes which are substantially collinear and overlap.

19. An autoinjector for injecting a formulation into a subject in need thereof, wherein the autoinjector comprises:
(a) a plunger rod comprising a first surface of the plunger rod,
(b) an energy source mechanism comprising an energy source and a trigger button;
(c) a damping mechanism comprising a second surface and a damping medium that is located between the first surface and the second surface; and
(d) a delivery section comprising a drug cartridge; and
wherein the autoinjector comprises at least one of the following characteristics:
(i) the damping mechanism damps out force and pressure spikes during delivery from the autoinjector,
(ii) using the autoinjector results in lower device failures such as breakage of the drug cartridges and high initial delivery rates of the formulation,
(iii) the damping mechanism reduces variability in delivery time and performance due to varying formulation properties, such as viscosity,
(iv) the damping mechanism improves reproducibility,
(v) the autoinjector is used with a wider range of formulations,
(vi) the damping mechanism has few or no additional components required other than the damping medium,
(vii) the damping is dependent at most linearly on a critical dimension of the damping mechanism,
(viii) the autoinjector is used as a multi-dose autoinjector and the autoinjector does not result in displacement of the damping medium,
(ix) the damping mechanism damps out force and pressure spikes during delivery of the formulation from the autoinjector, and
(x) the autoinjector is used as a multi-dose autoinjector and the autoinjector does not require that the damping medium be replaced or restored between doses.

20. (canceled)

21. The autoinjector of claim 19, wherein the damping medium has a viscosity between about 10,000 cP and about 500,000 cP.

22. (canceled)

23. (canceled)

24. (canceled)

25. (canceled)

26. The autoinjector of claim 19, wherein between about 20% and about 40% of the released energy is absorbed by the damping medium.

27. (canceled)

28. (canceled)

29. (canceled)

30. The autoinjector of claim 1, wherein the first surface and the second surface are in contact with the damping medium and wherein after applying the force on the trigger button, a relative motion between the first surface and second surface results in Couette flow that damps out force and pressure spikes during delivery from the autoinjector.

31. (canceled)

32. (canceled)

33. (canceled)

34. (canceled)

35. (canceled)

36. The autoinjector of claim 19, wherein a force is exerted by the energy source in the range of about 10 N to about 200 N.

37. The autoinjector of claim 19, wherein more than about 20% of the released energy is absorbed by the damping medium.

38. The autoinjector of claim 19, wherein between about 10% and about 90% of the released energy is absorbed by the damping medium.

39. (canceled)

40. (canceled)

41. (canceled)

42. (canceled)

43. (canceled)

44. The autoinjector of claim 15, wherein the liquid formulation comprises a drug and a carrier and wherein the formulation has a viscosity of at least about 1 cP.

45. (canceled)

46. (canceled)

47. (canceled)

48. (canceled)

49. (canceled)

50. (canceled)

51. (canceled)

52. A method of optimizing the autoinjector of claim 1 for use with a specific formulation, the method comprises:
(a) supplying a multiplicity of damping mechanism components each of which comprises a second surface, wherein each of the damping mechanism components can be installed in the autoinjector without making any other changes to the mechanical design of the autoinjector, and wherein the damping mechanism components in the multiplicity differ from each other in that when installed in the autoinjector they define a range of average first gaps between the first surface and the second surface;
(b) selecting one of the damping mechanisms;
(c) installing the damping mechanism selected in step (b) in the autoinjector; and
(d) applying a force on the trigger button which triggers a release of energy from the energy source to the plunger rod, wherein the plunger rod is configured to transmit between about 10% and about 90% of released energy to the drug cartridge, and wherein the transmission causes at least a portion of the formulation to be delivered from the drug cartridge to the subject.

53. The method of claim 52, wherein the optimization is based on at least one component of the list comprising: formulation viscosity, formulation volume, needle gauge, needle length, and delivery time.

54. The method of claim 52, wherein the optimization further comprises changing one or more of the parameters chosen from a list comprising: energy contained in the energy source, the power provided by the energy source, the viscosity of the damping medium, a second gap between two components of the autoinjector, volume of the drug cartridge, needle gauge, and needle length.

55. A method for operating the autoinjector of claim 6 wherein the method comprises the following steps:
(a) a patient or a caregiver presses the distal end of the housing with the orifice against a target injection site and presses the trigger button releasing a spring which drives the plunger rod forward,
(b) the plunger rod moves forward relative to the housing,
(c) Couette shear flow sets up in the damping medium,
(d) the plunger rod contacts the stopper and drives the stopper and the drug cartridge forward urging the needle through the orifice and into the target injection site,
(e) the drug cartridge bottoms out against the housing and stops, and
(f) the plunger rod advances the stopper through the drug cartridge under continued urging of the spring, delivering at least some of the formulation in the drug cartridge through the needle.

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)
